(19) United States
(12) Patent Application Publication
(10) Pub. No.: US 2022/0071522 A1
(43) Pub. Date: Mar. 10, 2022

(54) MEASUREMENT APPARATUS

(71) Applicant: OMRON HEALTHCARE Co., Ltd., Kyoto (JP)

(72) Inventors: Hideie HIRATA, Kyoto (JP); Toru DENO, Kyoto (JP); Takashi KITAMURA, Kyoto (JP); Takuya ITO, Kyoto (JP)

(21) Appl. No.: 17/448,303

(22) Filed: Sep. 21, 2021

Related U.S. Application Data
(63) Continuation of application No. PCT/JP2020/010439, filed on Mar. 11, 2020.

(30) Foreign Application Priority Data
Mar. 28, 2019 (JP) 2019-064216

(51) Int. Cl. A61B 5/145; G01D 9/00; G16H 40/63

(52) U.S. Cl. CPC A61B 5/14532; G16H 40/63; G01D 9/00

(57) ABSTRACT

To provide a measurement apparatus that can acquire measurement date/time information without having a clock in a measurement unit. A health information management system includes a blood pressure monitor that measures blood pressure information and includes an RTC that can acquire the count value at the time of each measurement, and a smart phone including a clock unit that can acquire date/time information, and the health information management system can perform at least information transmission from the blood pressure monitor to the smart phone. The measurement date/time of blood pressure information is specified based on a difference between the count value at the time of measurement and the count value at the time of information transmission from the blood pressure monitor to the smart phone, and date/time information acquired by the smart phone from the clock unit.

### CROSS-REFERENCE TO RELATED APPLICATIONS

This application is the U.S. national stage application filed pursuant to 35 U.S.C. 365(c) and 120 as a continuation of International Patent Application No. PCT/JP2020/010439, filed Mar. 11, 2020, which application claims priority to Japanese Patent Application No. 2019-064216, filed Mar. 28, 2019, which applications are incorporated herein by reference in their entireties.

### TECHNICAL FIELD

The present invention relates to a measurement apparatus.

### BACKGROUND ART

In recent years, it has become increasingly popular to use measurement apparatuses to measure personal health-related information (hereinafter referred to as health information), such as weight, blood pressure value, and an amount of activity, and to record and analyze the measurement results on information terminals such as smart phones for health management.

In this kind of health management, changes in the measurement results by a measurement apparatus over time is useful information. In this case, in addition to the measurement results, it is necessary to record the date/time information when the measurement is performed.

Patent Document 1 discloses a technique in which a biological information measurement apparatus provided with a real time clock (RTC) transmits the measured biological information and the time information supplied by the RTC to a data center.

### CITATION LIST

### Patent Literature

Patent Document 1: JP 2005-261710 A

### SUMMARY OF INVENTION

### Technical Problem

However, the RTC alone can only acquire the elapsed time after the power-on of the measurement apparatus. On the other hand, if a clock is installed in each measurement apparatus to record the measurement date/time, the cost will increase. Another problem is that when multiple types of health information are collected by multiple types of measurement apparatuses, it is necessary to synchronize the RTCs of each measurement apparatus.

In view of the above-mentioned known techniques, an object of the present invention is to provide a measurement apparatus that can acquire measurement date/time information without having a clock in a measurement unit.

### Solution to Problem

To solve the above-mentioned problems, a measurement apparatus according to the present invention includes a measurement unit including a timing unit configured to measure an elapsed time from predetermined timing as a count value, a first measurement result accumulation unit configured to plurally accumulate a first measurement result including measurement information acquired through measurement, identification information uniquely assigned to the measurement information in order of acquisition of the measurement information and the count value at a time of measurement, and a transmission unit configured to transmit the first measurement result accumulated in the first measurement result accumulation unit, a reception unit configured to receive the first measurement result from the measurement unit, a second measurement result accumulation unit configured to accumulate a second measurement result including at least date/time information at the time of the measurement and the measurement information included in the first measurement result received from the measurement unit, and a measurement date/time calculation unit configured to calculate a measurement date/time at which the measurement information is acquired by subtracting a difference between the count value at a reference date/time and the count value at the time of the measurement from the reference date/time for the first measurement result, the reference date/time being a time point when communication with the measurement unit is established.

Here, the above-mentioned measurement apparatus is not limited to a device configured as a unit, and may be a system independently composed of a plurality of devices. In addition, the measurement apparatus includes an apparatus including a plurality of measurement units.

The independent device including the measurement unit and the transmission unit includes body information measurement apparatuses such as weight scales, body composition meters, blood pressure monitors, heart rate monitors, and thermometers, activity measurement apparatuses such as pedometers and activity meters provided in various fitness equipment, environmental information measurement apparatuses such as temperature and humidity meters, sound level meters, and illuminometers, and various other measurement apparatuses. In addition, the above-mentioned measurement information includes body information such as weight, body fat percentage, blood pressure, heart rate (pulse), and body temperature, an amount of activity such as the number of steps, running distance, and heat consumption, environment information such as room temperature, humidity, noise level, and illuminance, and other values of various quantities depending on the measurement apparatus.

In addition, the independent device including the reception unit and the measurement date/time calculation unit includes portable information terminals such as smart phones, tablet terminals, and laptops, as well as stationary information terminals.

With the timing unit that measures the elapsed time from predetermined timing, the above-mentioned configuration can acquire measurement date/time information based on the count value acquired by the timing unit even without having a clock in the measurement unit. Since it is not necessary to provide a clock in the measurement unit, the cost of the measurement unit can be reduced, and in turn, the cost of the entire measurement apparatus can be reduced.

In addition, in the above-described measurement apparatus, the measurement unit may calculate the difference, and transmit the difference to the measurement date/time calculation unit.

In addition, in the above-described measurement apparatus, the difference may be calculated at the measurement date/time calculation unit on a basis of the count value at the reference date/time and the count value at the time of the measurement transmitted from the measurement unit.

In this manner, the processing load at the measurement unit can be reduced because the calculation of the difference is not performed at the measurement unit.

In addition, in the above-described measurement apparatus, the measurement unit may stores power recovery information related to a loss and a recovery of power at the measurement unit, and the measurement date/time calculation unit may determine whether the measurement information included in the first measurement result is acquired before the power loss on a basis of the power recovery information received from the measurement unit, and, when the measurement information is acquired before the power loss, the measurement date/time calculation unit may calculate the measurement date/time of the measurement information corresponding to the first measurement result by adding, to the measurement date/time included in the second measurement result that is finally accumulated in the second measurement result accumulated in the second measurement result accumulation unit and for which the measurement date/time has been calculated, a difference between the count value at the time of the measurement corresponding to the second measurement result that is finally accumulated and the count value at the time of the measurement corresponding to the first measurement result.

In this manner, with the timing unit that measures the elapsed time from predetermined timing, it is possible to acquire the measurement date/time information based on the count value acquired by the timing unit even without having a clock in the measurement unit. Since it is not necessary to provide a clock in the measurement unit, the cost of the measurement unit can be reduced, and in turn, the cost of the entire measurement apparatus can be reduced. Further, even in the case where the power of the measurement unit is lost and the elapsed time up to that point is cleared, the measurement date/time information can be acquired without providing a clock in a measurement unit.

Here, the loss and recovery of the power include a loss of the power due to detaching a battery from the measurement apparatus, and a recovery of the power due to attaching a battery to the measurement apparatus, i.e., replacement of a battery. In this case, the power recovery information corresponds to the battery replacement information, and a power recovery flag described later corresponds to a battery replacement flag. In addition, the loss and recovery of the power include a loss of the power due to pulling the power plug of the measurement apparatus out of the outlet, and a recovery of the power due to plugging of the power plug of the measurement apparatus into the outlet. Further, the loss and recovery of the power include a case where the power of the measurement apparatus whose power plug is plugged in the outlet is lost due to cut-off of power supply to the outlet due to power outage and the like, and the power of the measurement apparatus whose power plug is plugged in the outlet is recovered due to restart of power supply to the outlet.

In addition, in the above-described measurement apparatus, at a time of a first power recovery after the power loss, the measurement unit may assign a power recovery flag as power recovery information to all the first measurement results accumulated in the first measurement result accumulation unit, and the measurement date/time calculation unit may determine that the measurement information included in the first measurement result assigned with the power recovery flag has been acquired before the power loss.

In addition, in the above-described measurement apparatus, at a first power recovery after the power loss, the measurement unit may assign a power recovery flag that is the power recovery information to a latest first measurement result in the first measurement results accumulated in the first measurement result accumulation unit, the measurement unit may transmit the first measurement result accumulated in the first measurement result accumulation unit in order from a latest one, and the measurement date/time calculation unit may determine that the measurement information included in the first measurement result assigned with the power recovery flag and the first measurement result received after the first measurement result assigned with the power recovery flag has been acquired before the power loss.

In addition, in the above-described measurement apparatus, at a first power recovery after the power loss, the measurement unit may store, as reference identification information, the identification information included in the measurement information included in a latest first measurement result in the first measurement results accumulated in the first measurement result accumulation unit, and when it is indicated that the identification information assigned to the measurement result is acquired at a time point before the reference identification information as a result of comparison between the identification information assigned to each measurement information and the reference identification information at a time of transmission of the first measurement result, the measurement unit may assign a power recovery flag as the power recovery information to the measurement information and perform the transmission, and for a measurement result assigned with the power recovery flag, the measurement date/time calculation unit may determine that the measurement information that is received has been acquired before the power loss.

In addition, in the above-described measurement apparatus, at a first power recovery after the power loss, the measurement unit may store, as reference identification information, the identification information included in the measurement information included in a latest first measurement result in the first measurement results accumulated in the first measurement result accumulation unit, and when, at transmission of the first measurement result in descending order of the identification information from the latest first measurement result in the first measurement results accumulated in the first measurement result accumulation unit, the identification information included in the first measurement result to be transmitted is identical to the reference identification information as a result of comparison between the identification information included in the first measurement result to be transmitted and the reference identification information, the measurement unit may assign a power recovery flag that is the power recovery information to the first measurement result to be transmitted and perform the transmission, and the measurement date/time calculation unit may determine that the measurement information included in the first measurement result assigned with the power recovery flag and the first measurement result received after the first measurement result assigned with the power recovery flag has been acquired before the power loss.

In addition, in the above-described measurement apparatus, at a first power recovery after the power loss, the measurement unit may store, as reference identification information that is the power recovery information, the identification information of a latest first measurement result in the first measurement results accumulated in the first measurement result accumulation unit, and transmit the reference identification information at a time of transmission of the first measurement result, and when it is indicated that the identification information of the first measurement result that is received is acquired at a time point before the reference identification information as a result of comparison between the identification information and the reference identification information, the measurement date/time calculation unit may determine that the measurement information included in the first measurement result has been acquired before the power loss.

In addition, in the above-described measurement apparatus, when the count value at the time of the measurement corresponding to the second measurement result finally accumulated in the second measurement result accumulation unit is not accumulated in the first measurement result accumulation unit, or when it is indicated that the count value at the time of the measurement corresponding to the first measurement result is before the count value at a time of one previous measurement of the first measurement result, the measurement unit or the measurement date/time calculation unit may set the difference as invalid, and for the first measurement result that is acquired before the power loss and is measured on and after the measurement of the first measurement result whose difference is invalid, the measurement date/time calculation unit may set the measurement date/time as invalid and performs accumulation in the second measurement result accumulation unit.

In addition, the above-described measurement apparatus may further include a display data generation unit configured to display the measurement information in a time-series manner. for the second measurement result whose measurement date/time is invalid, the display data generation unit may generate a temporary graph in which the measurement information of the second measurement result assigned with the identification information that is successive is disposed in a time-series manner in accordance with the difference of the count value at the time of the measurement between the measurement information assigned with the identification information that is successive, and dispose the temporary graph such that a center in a time direction of the temporary graph matches a center in the time direction between the measurement dates/times that are not invalid immediately before and after the second measurement result whose measurement date/time is invalid.

In this manner, even for measurement information whose exact measurement date/time cannot be calculated, a time-series change relative to other measurement information whose measurement date/time has been specified.

In addition, in the above-described measurement apparatus, the measurement unit may store power recovery history information related to a history of a loss and a recovery of power at the measurement unit, the first measurement result accumulation unit may accumulate the first measurement result including the power recovery history information at the time of the measurement, the second measurement result accumulation unit may accumulate the first measurement result including the power recovery history information received from the measurement unit, and when the power recovery history information at a time of acquisition of the measurement information included in the first measurement result received from the measurement unit is identical to the power recovery history information that is latest in the power recovery history information, the measurement date/time calculation unit may calculate the measurement date/time at which the measurement information is acquired by subtracting the difference between the count value at the reference date/time and the count value at the time of the measurement from the reference date/time for the first measurement result, the reference date/time being a time point when a communication with the measurement unit is established.

In this manner, even in the case where the measurement unit has undergone the loss and recovery of the power, the measurement date/time information can be acquired based on the count value acquired by the timing unit for the first measurement result having the same history of the power loss and recovery as the latest history of the power loss and recovery, without having a clock providing in the measurement unit.

Here, the power recovery history information is information representing a history of a loss and recovery of the power, such as information about the number of times of loss and recovery of the power up to the current time, but is not limited thereto as long as it is information corresponding to a history of a loss and recovery of the power.

In addition, the loss and recovery of the power include a loss of the power due to detaching a battery from the measurement apparatus, and a recovery of the power due to attaching a battery to the measurement apparatus, i.e., replacement of a battery. In this case, the power recovery history information corresponds to the battery replacement history information. In addition, the loss and recovery of the power include a loss of the power due to pulling the power plug of the measurement apparatus out of the outlet, and a recovery of the power due to plugging of the power plug of the measurement apparatus into the outlet. Further, the loss and recovery of the power include a case where the power of the measurement apparatus whose power plug is plugged in the outlet is lost due to cut-off of power supply to the outlet due to power outage and the like, and the power of the measurement apparatus whose power plug is plugged in the outlet is recovered due to restart of power supply to the outlet.

In addition, in the above-described measurement apparatus, the measurement unit may calculate the difference, and transmit the difference to the measurement date/time calculation unit.

In addition, in the above-described measurement apparatus, the difference may be calculated at the measurement date/time calculation unit on a basis of the count value at the reference date/time and the count value at the time of the measurement transmitted from the measurement unit.

In this manner, the processing load at the measurement unit can be reduced because the calculation of the difference is not performed at the measurement unit.

In addition, in the above-described measurement apparatus, the measurement date/time calculation unit may determine a power recovery history at the time of the acquisition of the measurement information included in the first measurement result on a basis of the power recovery history information received from the measurement unit, and the measurement date/time calculation unit may calculate the measurement date/time of the measurement information corresponding to the first measurement result through addition or subtraction of the difference between the count value at the time of the measurement corresponding to the second measurement result and the count value at the time of the measurement corresponding to the first measurement result with respect to the measurement date/time included in any of the second measurement results having a power recovery history identical to the power recovery history that is determined.

In this manner, regardless of the number of the first measurement results that have not been transmitted from the measurement unit to the reception unit, or have not been received at the reception unit after being sent, the measurement date/time can be calculated as long as one second measurement result that has the same history of the power loss and recovery and in which valid measurement date/time is recorded in the second measurement result accumulation unit. In addition, since the differential processing of the count value is not performed, the processing load of the measurement unit is small.

In addition, in the above-described measurement apparatus, the measurement date/time calculation unit may determine the power recovery history at the time of the acquisition of the measurement information included in the first measurement result on the basis of the power recovery history information received from the measurement unit, and, when no valid measurement date/time has been calculated for any of the second measurement results having the power recovery history identical to the power recovery history that is determined, the measurement date/time calculation unit may set the measurement date/time as invalid and perform accumulation in the second measurement result accumulation unit for the first measurement result.

In addition, the above-described measurement apparatus may further include a display data generation unit configured to display the measurement information in a time-series manner. For the second measurement result whose measurement date/time is invalid, the display data generation unit may generate a temporary graph in which the measurement information of the second measurement result assigned with the identification information that is successive is disposed in a time-series manner in accordance with the difference of the count value at the time of the measurement between the measurement information assigned with the identification information that is successive, and dispose the temporary graph such that a center in a time direction of the temporary graph matches a center in the time direction between the measurement dates/times that are not invalid immediately before and after the second measurement result whose measurement date/time is invalid.

In this manner, even for measurement information whose exact measurement date/time cannot be calculated, a time-series change relative to other measurement information whose measurement date/time has been specified.

In addition, in the above-described measurement apparatus, when there is a plurality of the power recovery histories in which the measurement date/time is invalid for all the second measurement results having the power recovery histories identical to each other, the display data generation unit may generate a temporary graph in which the measurement information of the second measurement result assigned with the identification information that is successive is disposed in a time-series manner in accordance with the difference of the count value at the time of the measurement between the measurement information assigned with the identification information that is successive for the power recovery histories in which the measurement date/time is invalid for all the second measurement results, divide the time direction between the measurement dates/times that are not invalid immediately before and after the measurement result whose measurement date/time is invalid into a plurality of time intervals for each power recovery history, and dispose the temporary graph such that a center in a time direction of the temporary graph of the second measurement result whose measurement date/time is invalid of each power recovery history matches a center of the time interval corresponding to each power recovery history.

In this manner, even in the case where a plurality of pieces of power recovery history information in which the measurement date/time is invalid for all the second measurement results having the same power recovery history information, a time-series change relative to other measurement information whose measurement date/time has been specified can be determined for the measurement information whose exact measurement date/time cannot be calculated.

### Advantageous Effects of Invention

According to the present invention, it is possible to provide a measurement apparatus that can acquire measurement date/time information without having a clock.

### BRIEF DESCRIPTION OF DRAWINGS

FIG. 1 is a block diagram schematically illustrating an example of a configuration of a health information management system according to a first embodiment.

FIG. 2 is a diagram illustrating an example of a display screen of a blood pressure monitor of the health information management system according to the first embodiment.

FIG. 3 is a diagram illustrating an example of a screen display of a smart phone of the health information management system according to the first embodiment.

FIG. 4 is a flowchart illustrating a flow of measurement and a recording process of a measurement result of the blood pressure monitor of the health information management system according to the first embodiment.

FIG. 5 is a flowchart illustrating a flow of a normal process of transmitting a measurement result to the smart phone at the blood pressure monitor of the health information management system according to the first embodiment.

FIG. 6 is a flowchart illustrating a flow from measurement to a measurement result transmission process to the smart phone at the blood pressure monitor of the health information management system according to the first embodiment.

FIG. 7 is a flowchart illustrating a flow of a process of transmitting a measurement result from the blood pressure monitor to the smart phone in the health information management system according to the first embodiment.

FIG. 8A and FIG. 8B are diagrams illustrating an example of a data configuration of a measurement result recording unit in the blood pressure monitor of the health information management system according to the first embodiment.

FIG. 9 is a flowchart illustrating a flow of a process of receiving measurement information at the smart phone of the health information management system according to the first embodiment.

FIG. 10 is a flowchart illustrating a flow of a process of specifying a measurement date/time at the smart phone of the health information management system according to the first embodiment.

FIG. 11 is a diagram illustrating an example of a data configuration of the measurement result recording unit in the smart phone of the health information management system according to the first embodiment.

FIG. 12A and FIG. 12B are diagrams illustrating an example of a data configuration of a measurement result recording unit in a blood pressure monitor of a health information management system according to a case 2 of the first embodiment.

FIG. 13 is a diagram illustrating an example of a data configuration of a measurement result recording unit in a smart phone of the health information management system according to the case 2 of the first embodiment.

FIG. 14 is a diagram illustrating an example of a display of a change with time of blood pressure information in the smart phone of the health information management system according to the case 2 of the first embodiment.

FIG. 15A and FIG. 15B are diagrams illustrating an example of a data configuration of a measurement result recording unit in a blood pressure monitor a health information management system according to a case 3 of the first embodiment.

FIG. 16 is a diagram illustrating an example of a data configuration of a measurement result recording unit in a smart phone of the health information management system according to the case 3 of the first embodiment.

FIG. 17 is a flowchart illustrating a flow of a process of transmitting a measurement result from a blood pressure monitor to a smart phone in a health information management system according to a second embodiment.

FIG. 18 is a flowchart illustrating a flow of a process of specifying a measurement date/time at the smart phone of the health information management system according to the second embodiment.

FIG. 19 is a flowchart illustrating a flow of a process of transmitting a measurement result from a blood pressure monitor to a smart phone of a health information management system according to a third embodiment.

FIG. 20 is a flowchart illustrating a flow of a process of receiving measurement information at the smart phone of the health information management system according to the third embodiment.

FIG. 21 is a flowchart illustrating a flow of a process of specifying a measurement date/time at the smart phone of the health information management system according to the third embodiment.

FIG. 22 is a flowchart illustrating a flow of a process of transmitting a measurement result from a blood pressure monitor to a smart phone in a health information management system according to a fourth embodiment.

FIG. 23 is a flowchart illustrating a flow of a process of receiving measurement information at the smart phone of the health information management system according to the fourth embodiment.

FIG. 24 is a flowchart illustrating a flow of a process of specifying a measurement date/time at the smart phone of the health information management system according to the fourth embodiment.

FIG. 25 is a flowchart illustrating a flow of measurement and a recording process of a measurement result at a blood pressure monitor of a health information management system according to a fifth embodiment.

FIG. 26 is a flowchart illustrating a flow of a process of transmitting a measurement result from the blood pressure monitor to the smart phone in the health information management system according to the fifth embodiment.

FIG. 27 is a flowchart illustrating a flow of a process of receiving measurement information at the smart phone of the health information management system according to the fifth embodiment.

FIG. 28 is a flowchart illustrating a flow of a process of specifying a measurement date/time at the smart phone of the health information management system according to the fifth embodiment.

FIG. 29 are diagrams illustrating an example of a data configuration of a measurement result at the blood pressure monitor and an example of a measurement date/time specifying process at the smart phone according to the fifth embodiment.

FIG. 30 are diagrams illustrating another example of a data configuration of a measurement result at a blood pressure monitor and another example of a measurement date/time specifying process at the smart phone according to the fifth embodiment.

FIG. 31 is a diagram illustrating an example of a display of a change with time of blood pressure information at a smart phone of a health information management system according to a case 2 of the fifth embodiment.

### DESCRIPTION OF EMBODIMENTS

Embodiments of the present invention will be specifically described below with reference to the drawings.

### First Embodiment

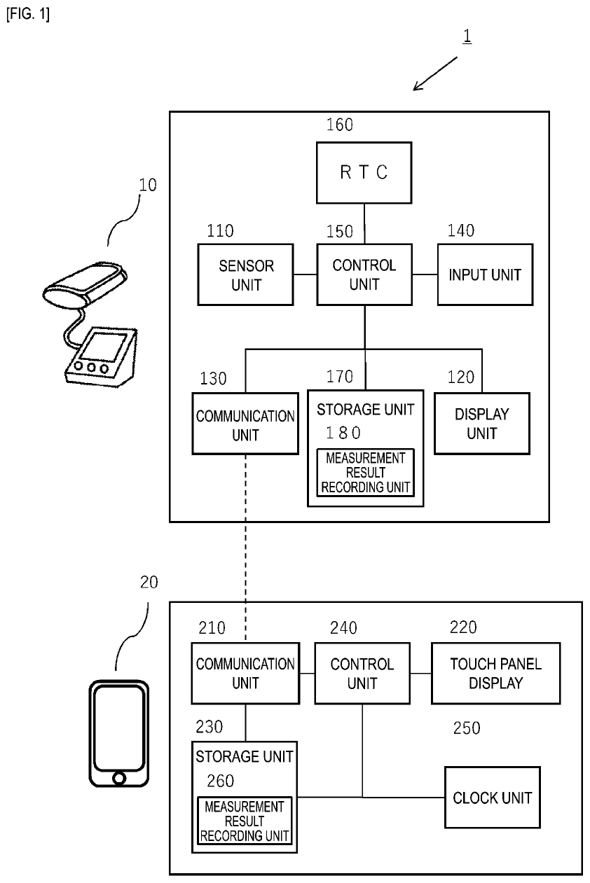

First, with reference to FIG. 1 to FIG. 16, an example of an embodiment of the present invention will be described below. It should be noted that the dimension, material, shape, relative arrangement and the like of the components described in the present embodiment are not intended to limit the scope of this invention to them alone, unless otherwise stated.

System Configuration

FIG. 1 is a schematic view illustrating an example of a configuration of a health information management system 1 that is a measurement apparatus according to the present embodiment. As illustrated in FIG. 1, the health information management system 1 includes a blood pressure monitor 10 as an example of a measurement unit, and a smart phone 20 including a measurement date/time calculation unit, and the blood pressure monitor 10 and the smart phone 20 are configured to be able to communicate with each other via near-field wireless communication. While the wireless communication method is not limited, it is possible to employ methods such as Bluetooth (trade name), infrared communication, and ultrasound information transmission, for example.

Blood Pressure Monitor

The blood pressure monitor 10 in the present embodiment is a measurement apparatus that measures the user's blood pressure by the so-called oscillometric method, and includes a sensor unit 110, a display unit 120, a communication unit 130, an input unit 140, a control unit 150, an RTC 160, and a storage unit 170 as illustrated in FIG. 1.

The sensor unit 110 includes a pressure sensor disposed at a cuff portion of the blood pressure monitor 10, and detects pulse waves from the user's blood vessel under a proper cuff pressure. The blood pressure monitor 10 in the present embodiment can measure pulses in addition to the systolic blood pressure and diastolic blood pressure on the basis of pulse waves detected by the sensor unit. In the following description, the values of the systolic blood pressure, diastolic blood pressure, and pulse are collectively referred to as blood pressure information. In the present embodiment, blood pressure information corresponds to measurement information.

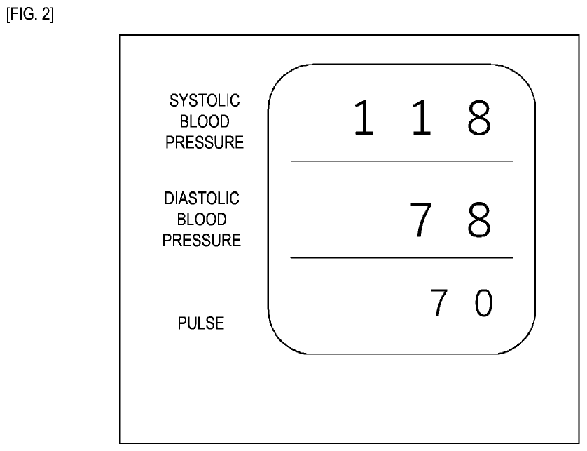

The display unit 120, which is formed of a liquid crystal display or the like for example, displays calculated blood pressure information. FIG. 2 illustrates an example of a display screen of the display unit 120.

The communication unit 130 is a communication antenna that transmits and receives signals used in near-field wireless communication as electromagnetic waves, and may employ desired publicly known techniques. The communication unit 130 transmits information including the inherent identification number (such as the serial number) of each blood pressure monitor, and the measured blood pressure information. It is to be noted that in the present embodiment, the communication unit 130 corresponds to a transmission unit.

The input unit 140 is an input means, such as a button and a touch panel display, that receives input from the user, and receives various operations such as ON/OFF of the power, the start of the measurement and item selection from the user.

The control unit 150 is a means that manages the control of the blood pressure monitor 10, and includes a central processing unit (CPU) and the like, for example. When receiving a measurement start instruction from the user through the input unit 140, the control unit 150 pressurizes the cuff, and calculates the blood pressure information on the basis of the pulse waves detected by the sensor unit 110 under an appropriate cuff pressure. Then, the calculated value is displayed on the display unit 120. In addition, each component of the blood pressure monitor 10 is controlled so as to execute processes in accordance with the user operation through the input unit 140.

The RTC 160 is a real time clock, which is a timer that counts up from predetermined timing such as power-on, and indicates the elapsed time from power-on. When the battery is replaced, the count value of the RTC 160 is cleared and counted up again from 0. In the present embodiment, the RTC 160 corresponds to a timing unit. In the embodiments including the present embodiment, a battery replacement is described as an example of the loss and recovery of the power, which causes the situation where the count value of the RTC 160 is cleared and counted up again from 0. In this case, the loss of the power corresponds to a removal of the battery from the blood pressure monitor 10, and the recovery of the power corresponds to attachment of the battery to the blood pressure monitor 10. In the case where the blood pressure monitor 10 receives power supply from the power plug plugged in the outlet, the loss of the power corresponds to pulling out of the power plug from the outlet, and the recovery of the power corresponds to plugging of the power plug into the outlet. In addition, in the state where the power plug of the blood pressure monitor 10 is plugged in the outlet, the stop of power supply through the outlet due to power outage and the like corresponds to the loss of the power, and the restart of power supply through the outlet corresponds to the recovery of the power.

The storage unit 170 includes, for example, a long-term storage medium such as a flash memory in addition to a main storage device such as a RAM, and stores various information such as an application program and measurement information.

In a predetermined region of the storage unit 170, the sequence number representing the number of times of the measurements by the blood pressure monitor 10 is stored. The sequence number is a number that is uniquely assigned in ascending order in the order of measurement. It is updated every time a measurement is performed, and the above-described region holds the latest sequence number representing the number of times of the measurements at the current time point. The sequence number need only be able to identify the order of the measurement of the assigned measurement information, and may be assigned in descending order of measurement as well as in ascending order of measurement.

A measurement result recording unit 180 is a nonvolatile memory included in the storage unit 170, and records information such as the blood pressure information measured by the sensor unit 110, the count value of the RTC and the sequence number assigned to the measurement. In the present embodiment, the measurement result recording unit 180 corresponds to a first measurement result accumulation unit. In addition, in the present embodiment, the information such as the blood pressure information measured by the sensor unit 110 and the count value of the RTC corresponds to a first measurement result.

Smart Phone

As illustrated in FIG. 1, the smart phone 20 includes a communication unit 210, a touch panel display 220, a storage unit 230, a control unit 240, and a clock unit 250.

The communication unit 210 is a communication antenna that transmits and receives electromagnetic waves including electromagnetic waves for short-range wireless communication, which receives measurement information transmitted from the blood pressure monitor 10, and transmits and receives radio waves to/from other electronic devices and base stations. In the present embodiment, the communication unit 210 corresponds to a reception unit.

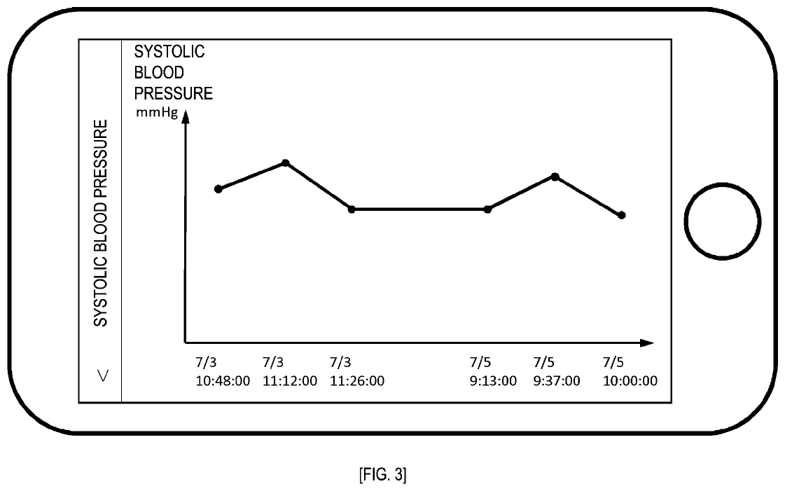

The touch panel display 220 serves as a display means and an input means, and displays the blood pressure information received by the communication unit 210. FIG. 3 illustrates an example of a transition of a 3o measured systolic blood pressure displayed along the time-series of the measurement date/time on the touch panel display 220. In addition, operations from the user are received through various images for input. It is to be noted that in the present embodiment, the touch panel display 220 corresponds to a second display means and an input means.

The storage unit 230 includes, for example, a long-term storage medium such as a flash memory in addition to a main storage device such as a RAM, and stores various information such as an application program and measurement information.

A measurement result recording unit 260 is a nonvolatile memory included in the storage unit 230, and records information such as blood pressure information received from the blood pressure monitor 10 and the measurement date/time of the blood pressure information. In the present embodiment, the measurement result recording unit 260 corresponds to a second measurement result accumulation unit. In addition, in the present embodiment, the information such as blood pressure information and the measurement date/time of the blood pressure information corresponds to a second measurement result.

The control unit 240 is a means that manages the control of the smart phone, which, for example, includes a CPU and the like and executes various programs stored in the storage unit 230 to serve functions of them. In the present embodiment, the control unit 240 corresponds to a measurement date/time calculation unit and a display data generation unit.

The clock unit 250 has a timing function, and holds the current date/time information.

Specification of Measurement Date/Time

Next, a method of specifying the measurement date/time using the blood pressure monitor 10 and the smart phone 20 will be described.

Measurement of Blood Pressure Information Using Blood Pressure Monitor

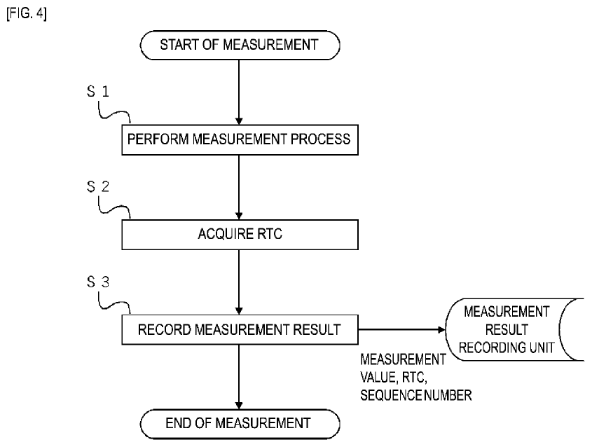

FIG. 4 is a flowchart illustrating a processing procedure of measurement of the blood pressure information and recording of measurement results using the blood pressure monitor 10.

The user wraps the cuff around the arm to prepare for the measurement, and presses the measurement start button included in the input unit 140 of the blood pressure monitor 10. When detecting the pressing of the measurement start button, the control unit 150 performs a predetermined blood pressure information measurement process (step S1).

Next, from the RTC 160, the control unit 150 acquires the count value at the time of measurement, i.e., the elapsed time from power ON (step S2). In the measurement, there is a certain time duration, and therefore the timing when the count value is to be acquired as the time of measurement may be appropriately set. For example, the count value at the start of measurement may be set as the count value at the time of measurement, but the present invention is not limited to this.

Next, the control unit 150 stores, in the measurement result recording unit 180, the blood pressure information as the measurement result and the count value of the RTC as the elapsed time together with a sequence number corresponding to the measurement (step S3).

Transmission of Measurement Results to Smart Phone

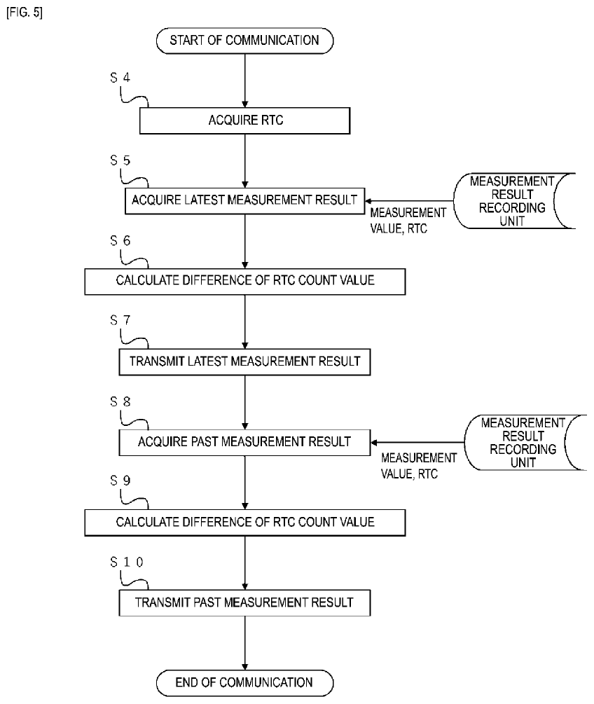

FIG. 5 is a flowchart illustrating a flow of a process of transmitting measurement results from the blood pressure monitor 10 to the smart phone 20.

It is desirable to transmit the measurement result for each measurement so that the smart phone 20 can hold the latest measurement result at all times.

This process of transmitting measurement results may be automatically performed following the measurement of the blood pressure information illustrated in FIG. 4, or may be performed when the user presses a transmission button.

First, the control unit 150 establishes communication with the smart phone 20 through the communication unit 130. When the communication between the blood pressure monitor 10 and the smart phone 20 is established, the control unit 150 acquires the count value of the RTC as of the transmission time (step S4).

Next, the control unit 150 acquires, from the measurement result recording unit 180, the latest measurement result, i.e., the measurement value and the count value of the RTC at the time of measurement with the largest sequence number (step S5).

Next, the control unit 150 calculates the difference between the count value of the RTC as of the transmission time acquired at step S4 and the count value of the RTC at the time of measurement acquired at step S5 (step S6).

Then, the control unit 150 transmits, to the smart phone 20 through the communication unit 130, the latest measurement result including the measurement value with the largest sequence number acquired at step S5 and the difference of the count value of the RTC calculated at step S6 (step S7).

Next, the control unit 150 acquires, from the measurement result recording unit 180, a past measurement result, i.e., the measurement value and the count value of the RTC at the time of measurement with the second largest sequence number (step S8).

Next, the control unit 150 calculates the difference between the count value of the RTC as of the transmission time acquired at step S4 and the count value of the RTC at the time of measurement acquired at step S8 (step S9).

Then, the control unit 150 transmits, to the smart phone 20 through the communication unit 130, the past measurement result including the measurement value with the second largest sequence number acquired at step S8 and the difference of the count value of the RTC calculated at step S9 (step S10). Here, the transmission may be performed by acquiring the past measurement results one by one, or by collectively acquiring the past measurement results. In addition, all measurement results accumulated in the measurement result recording unit 180 may be transmitted to the smart phone 20 by repeating acquisition and transmission of one or a plurality of measurement results.

In this manner, by transmitting the latest measurement result and the past measurement result from the blood pressure monitor 10 to the smart phone 20 for each measurement of blood pressure information as a normal process, the measurement date/time can be calculated at the smart phone 20 by subtracting the difference between the count value of the RTC at the time of transmission and the count value of the RTC at the time of the measurement from the date/time information as of the transmission time for the latest measurement result after the battery replacement, even when the battery is replaced.

However, in the case where only communication from the blood pressure monitor 10 to the smart phone 20 in one direction is performed, the blood pressure monitor 10 side cannot recognize a situation where no reception has been made at the smart phone 20 after a transmission from the blood pressure monitor 10. In addition, the latest measurement result or the past measurement result may not be transmitted due to some trouble in the blood pressure monitor 10.

Even in such an exceptional case, with the process described later, the measurement date/time can be specified on the smart phone 20 side for measurement results measured using the blood pressure monitor 10 with no clock function.

Flow of Processes from Measurement of Blood Pressure Information at Blood Pressure Monitor to Process of Transmitting Measurement Results to Smart Phone

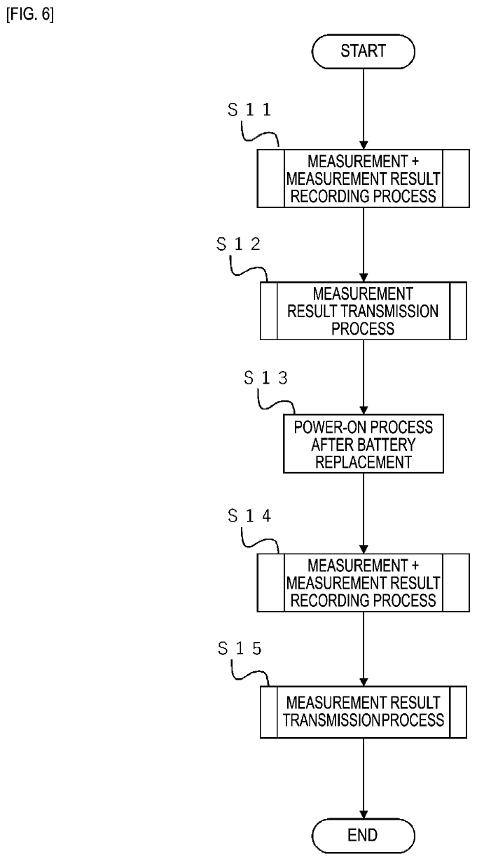

FIG. 6 is a flowchart illustrating an example of a flow of processes in the blood pressure monitor 10, from a measurement of blood pressure information to a process of transmitting measurement results to the smart phone 20.

When the user measures the blood pressure information using the blood pressure monitor 10, the control unit 150 performs the measurement process and measurement result recording process illustrated in FIG. 4 (step S11).

Next, the control unit 150 of the blood pressure monitor 10 performs the process of transmitting measurement results illustrated in FIG. 5 at predetermined timing (step S12).

When the user turns on the power for the first time after replacement of the battery of the blood pressure monitor 10, the control unit 150 performs a predetermined power-on process (step S13).

Here, the latest sequence number of the sequence numbers assigned to the respective measurement results recorded in a predetermined region (hereinafter also referred to as “measurement result memory”) of the measurement result recording unit 180 composed of a nonvolatile memory is stored in a predetermined region of the measurement result recording unit 180 as a pre-battery replacement sequence number. Regarding the region where the pre-battery replacement sequence number is to be stored, it is not limited to the measurement result recording unit 180, and it may be stored in another region of the nonvolatile memory included in the storage unit 170. At the time of power-on, the control unit 150 performs other various initialization processes, which are known processes and therefore are not elaborated. In the present embodiment, the sequence number corresponds to identification information, and the pre-battery replacement sequence number corresponds to battery replacement information and reference identification information.

In this manner, by storing only the pre-battery replacement sequence number, the load of the process of the first power-on after the battery replacement is reduced, and the time required for the process can be shortened.

When the user turns on the power after the battery replacement and measures blood pressure information, the control unit 150 performs the measurement process and measurement result recording process illustrated in FIG. 4 (step S14).

Next, at predetermined timing, the control unit 150 performs a process of transmitting measurement results from the blood pressure monitor 10 to the smart phone 20 described later (step S15). The timing when the blood pressure monitor 10 transmits the measurement result to the smart phone 20 may be appropriately set such that the measurement results including the past results are transmitted each time when the blood pressure information is measured. Alternatively, the timing may be timing when the user presses the transmission button included in the input unit 140, or timing when control unit 150 determines power-on, elapse of a predetermined time, execution of a predetermined number measurements, and the like.

As the above-described power-on process after the battery replacement, it is possible to set, to 1, a battery replacement flag indicating whether or not the battery has been replaced for all the measurement results recorded in the measurement result memory at the time of power-on. In this manner, in comparison with the case where the battery replacement flag is set based on the pre-battery replacement sequence number at the time of transmission of measurement results, the load of the process at the time of transmission is reduced, and the time required for the process can be shortened.

In addition, in the above-described power-on process after the battery replacement, it is possible to set the battery replacement flag to 1 for only the latest measurement result, i.e., the measurement result assigned with the latest sequence number, of the measurement results stored in the measurement result memory at the time of power-on. In this manner, in comparison with the case where the battery replacement flag is set to 1 for all measurement results, the load of the process is reduced, and the time required for the process can be shortened.

FIG. 6 illustrates an example of a flow of processes from the measurement of blood pressure information to the process of transmitting measurement results to the smart phone 20 in the blood pressure monitor 10, but this is not limitative. The measurement process and the measurement result recording process at step S11 and step S14 in FIG. 6 are typically repeated multiple times. In addition, while the battery replacement is performed one time before the transmission of the measurement result in FIG. 6, the battery replacement may be performed multiple times. One or multiple measurements of blood pressure information may be performed between multiple battery replacements. In addition, after the power-on process after the battery replacement is performed, the measurement result may be transmitted without measuring the blood pressure information.

Transmission of Measurement Result from Blood Pressure Monitor to Smart Phone

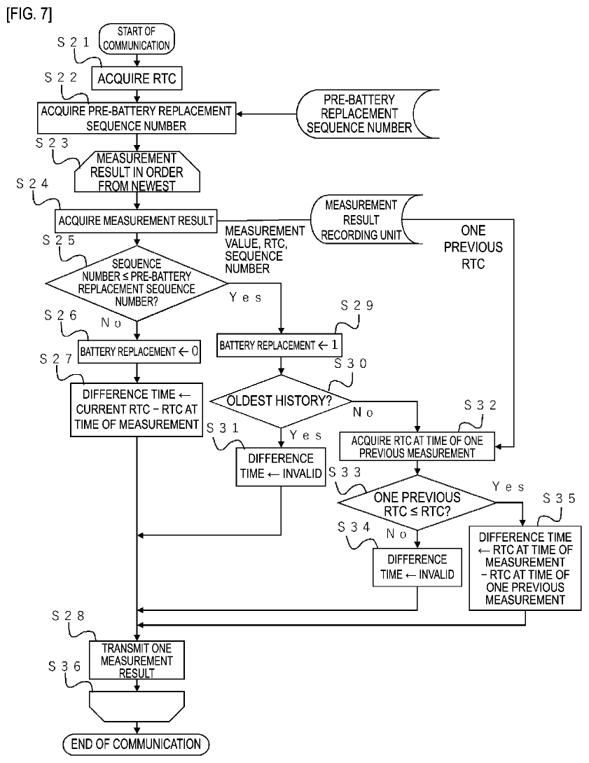

FIG. 7 is a flowchart illustrating a procedure of a process of transmitting data including a measurement result from the blood pressure monitor 10 to the smart phone 20.

First, the control unit 150 establishes communication with the smart phone 20 through the communication unit 130. When the communication between the blood pressure monitor 10 and the smart phone 20 is established, the control unit 150 acquires the count value of the RTC as of the transmission time (step S21). In the present embodiment, the time point as of the transmission time when the communication between the blood pressure monitor 10 and the smart phone 20 is established corresponds to the reference date/time.

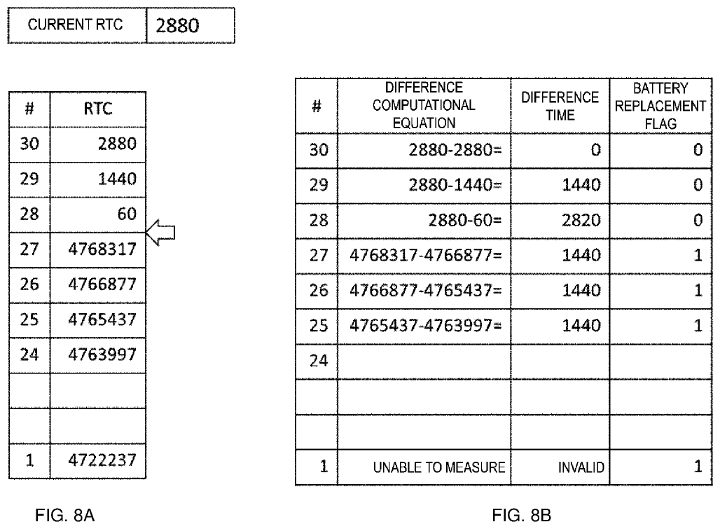

Next, the control unit 150 acquires a pre-battery replacement sequence number from a predetermined region of a nonvolatile memory (step S22). Now, a data configuration of the measurement result recording unit 180 is described with reference to FIG. 8A and FIG. 8B. FIG. 8A and FIG. 8B are diagrams schematically illustrating a configuration of data recorded in the measurement result recording unit 180. Here, the measurement result not illustrated in the drawing is associated with a sequence number assigned in ascending order (stored in the column of the title line “#” in FIG. 8A). Further, in the measurement result recording unit 180, the count value of the RTC at the time of measurement (stored in the column of the title line “RTC” in FIG. 8A) is associated with each measurement result. As illustrated in FIG. 8A, the count value at the time of measurement of the measurement result to which the sequence number 1 is assigned is 4722237 seconds (hereinafter, for the count values, the units are omitted and only numerical values are indicated). The larger the sequence number of the measurement result, the later the measurement timing, and accordingly the larger the count value of the RTC, which is the elapsed time from power ON. It should be noted that when the battery is replaced, the count value of the RTC is cleared and reset to 0. As described above, in the blood pressure monitor 10, at the time of power-on after the battery replacement, the latest (maximum) sequence number at that time point is stored as the pre-battery replacement sequence number. In FIG. 8A, the battery is replaced between measurements of sequence numbers 27 and 28, and accordingly the sequence number 27 is stored as the pre-battery replacement sequence number.

With reference to FIG. 8A, at step S22, “27” is acquired as the pre-battery replacement sequence number.

Next, in order from the newest measurement result (step S23), i.e., in descending order from the one with the largest sequence number, the control unit 150 acquires the measurement result and the count value of the RTC at the time of measurement from the measurement result recording unit 180 (step S24).

Then, the control unit 150 determines whether the sequence number of the measurement result acquired at step S23 is not greater than the pre-battery replacement sequence number acquired at step S22 (step S25).

When it is determined to be No at step S25, i.e., when the sequence number of the measurement result acquired at step S24 is greater than the pre-battery replacement sequence number acquired at step S22, the control unit 150 sets the battery replacement flag to 0 because it is the measurement result after the battery replacement (step S26). In the example illustrated in FIG. 8B, the measurement results of the sequence numbers 28 to 30 are the results of the post-battery replacement, 0 indicating OFF is set to the battery replacement flag. Then, as a difference time, the control unit 150 sets a value obtained by subtracting the count value of the RTC at the time of measurement of the measurement result acquired at step S24 from the count value of the RTC as of the transmission time acquired at step S21 (step S27). In the example illustrated in FIG. 8B, for the measurement result of the sequence number 30, 0, as a value obtained by subtracting 2880, which is the count value of the RTC at the time of measurement, from the count value 2880 of the RTC as of the transmission time is set as the difference time in accordance with the difference computational equation of step S27. Likewise, for the measurement result of the sequence number 29, 1440, which is a value obtained by subtracting 1440 from 2880, is set as the difference time. Likewise, for the measurement result of the sequence number 28, 2820, which is a value obtained by subtracting 60 from 2880, is set as the difference time. Then, through the communication unit 130, the control unit 150 transmits, to the smart phone 20, the blood pressure information, the sequence number, the value of the battery replacement flag set at step S26, and the value of the difference time set at step S27 as the measurement result for one case (step S28).

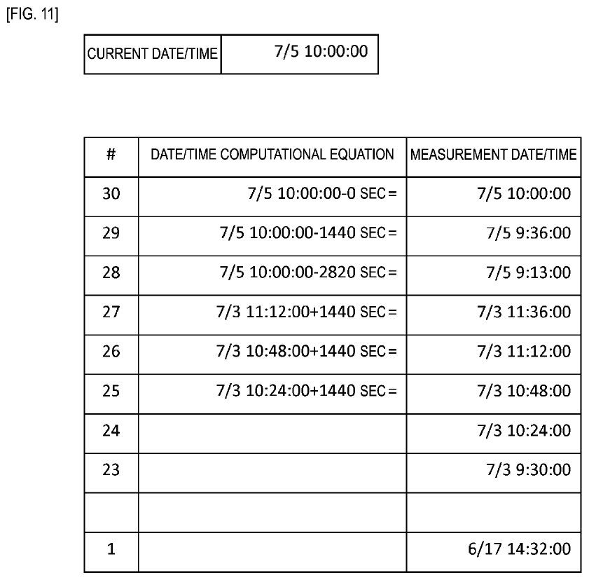

When it is determined to be Yes at step S25, i.e., when the sequence number of the measurement result acquired at step S24 is not greater than the pre-battery replacement sequence number acquired at step S22, the control unit 150 sets the battery replacement flag to 1 because it is the pre-battery replacement measurement result (step S29). In the example illustrated in FIG. 8B, the measurement results of the sequence numbers 1 to 27 are pre-battery replacement results, and therefore 1indicating ON is set to the battery replacement flag. The measurement results of the sequence number 24 or smaller have already been transmitted to the smart phone 20, and therefore the measurement dates/times of these measurement results are specified on the smart phone 20 as illustrated in FIG. 11. Then, the control unit 150 determines whether the measurement result is the oldest history (step S30). Here, whether it is the oldest history is determined based on whether a measurement result with a smaller sequence number is stored in the measurement result recording unit 180.

When it is determined to be Yes at step S30, the control unit 150 sets “invalid” as a difference time (step S31), and the process proceeds to step S28. In the example illustrated in FIG. 8A, the measurement result of the sequence number 1 is the oldest history, and is therefore set as “invalid” as the difference time.

When it is determined to be No at step S30, the control unit 150 acquires the count value of the RTC at the time of one previous measurement from the measurement result recording unit 180 (step S32).

Then, the control unit 150 determines whether the count value of the

RTC at the time of one previous measurement acquired at step S32 is not greater than the count value of the RTC acquired at step S24 (step S33).

When it is determined to be No at step S33, the control unit 150 sets “invalid” as the difference time (step S34), and the process proceeds to step S28. Normally, the count value of the RTC at the time of one previous measurement acquired at step S32 does not exceed the count value of the RTC acquired at step S24, and therefore in such a case, the “invalid” is set as the difference time because any of the measurement results is considered to be an error.

When it is determined to be Yes at step S33, the control unit 150 sets, as a difference time, a value obtained by subtracting the count value of the RTC at the time of one previous measurement acquired at step S32 from the count value of the RTC at the time of measurement acquired at step S24 (step S35), and the process proceeds to step S28. In the example illustrated in FIG. 8B, for the measurement result of the sequence number 27, 1440, which is a value obtained by subtracting 4766877, which is the count value of the RTC of the measurement result of the sequence number 26 as the RTC at the time of one previous measurement from 4768317, which is the count value of the RTC of the measurement result of the sequence number 27 is set as the difference time in accordance with the difference computational equation of step S35. Likewise, for the measurement result of the sequence number 26, 1440 is set as the difference time. Likewise, for the measurement result of the sequence number 25, 1440 is set as the difference time.

After repeating the processes from step S24 to step S28 in the order from the new measurement result for all measurement results (step S36), the control unit 150 disconnects and terminates the communication with the smart phone 20 through the communication unit 130.

Measurement Date/Time Specifying Process at Smart Phone Having Received Measurement Results from Blood Pressure Monitor

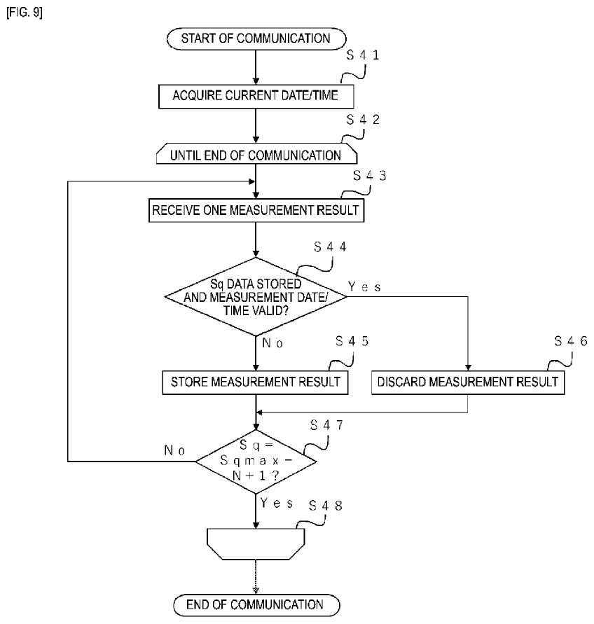

FIG. 9 is a flowchart illustrating a procedure of a process of receiving data including a measurement result from the blood pressure monitor 10 at the smart phone 20.

When the communication with the blood pressure monitor 10 is established and started, the control unit 240 acquires the current date/time information from the clock unit 250 (step S41).

Next, until the communication with the blood pressure monitor 10 is completed (step S42), the control unit 240 receives the measurement results one by one through the communication unit 210 (step S43). At this time, the measurement result is transmitted from the blood pressure monitor 10 in the order from one with the largest sequence number, and thus the smart phone 20 receives the measurement result in the order from one with the largest sequence number.

The control unit 240 determines whether the measurement result of the received sequence number (Sq) is stored in the measurement result recording unit 260, and a valid measurement date/time is specified for the measurement result (step S44).

When it is determined to be No at step S44, the measurement result is stored in the measurement result recording unit 260 (step S45), and the process proceeds to step S47.

When it is determined to be Yes at step S44, the measurement result is discarded without storing it (step S46), and the process proceeds to step S47.

At step S47, whether the sequence number Sq of the measurement result received at step S43 is Sqmax-N+1 is determined. Here, Sqmax is a measurement result with the largest sequence number in the measurement results received from the blood pressure monitor 10. N represents the number of cases that can be recorded in the measurement result memory of the measurement result recording unit 180 of the blood pressure monitor 10. Regarding Sqmax, because the measurement results are transmitted from the blood pressure monitor 10 in descending order from the largest sequence number, the sequence number of the measurement result received first may be stored in a predetermined region of the measurement result recording unit 260, or it may be transmitted from the blood pressure monitor 10 separately from the measurement result. In addition, the value of N may be transmitted from the blood pressure monitor 10 to the smart phone 20 and stored in a predetermined region of the storage unit 230 when making the blood pressure monitor 10 and the smart phone 20 recognizable through pairing and the like, but the method of acquiring the value of N is not limited to this.

When it is determined to be Yes at step S47, the communication is terminated.

When it is determined to be No at step S47, the process is returned to step S43, and the next measurement result is received.

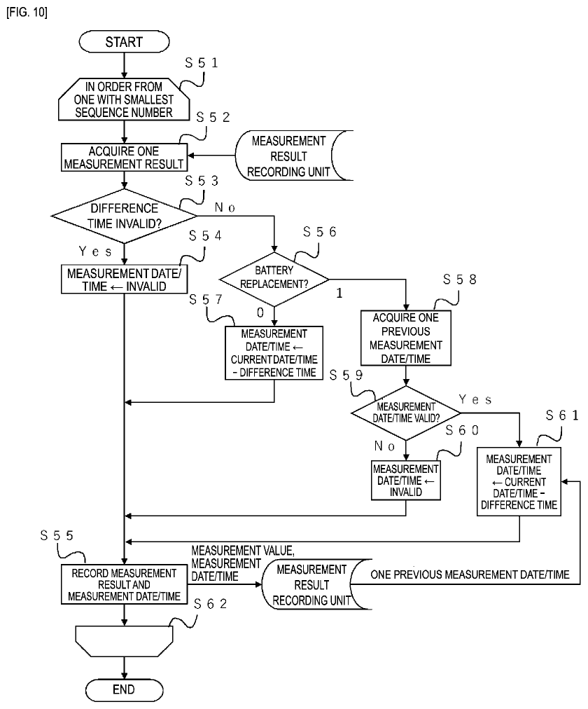

FIG. 10 is a flowchart illustrating a procedure of a process of specifying the measurement date/time to be executed at the smart phone 20 for the measurement result received in accordance with FIG. 9. A measurement date/time specifying process at the control unit 240 will be described below with reference to FIG. 10.

The control unit 240 repeats a process in the order from the one with the smallest sequence number for the measurement results received at step S43 to step S46 (step S51).

First, from the measurement result recording unit 260, the control unit 240 acquires one measurement result with the smallest sequence number in the measurement results received at step S43 to step S46 (step S52). The control unit 240 determines whether the data set as the difference time is “invalid” in the measurement result acquired at step S52 (step S53).

When it is determined to be Yes at step S53, the control unit 240 sets “invalid” as the measurement date/time (step S54). Then, the control unit 240 records the measurement result and the measurement date/time in the measurement result recording unit 260 (step S55).

When it is determined to be No at step S53, the control unit 240 determines whether the battery has been replaced (step S56). Here, the control unit 240 determines whether the battery has been replaced on the basis of the value of the battery replacement flag included in the measurement result received from the blood pressure monitor 10.

When it is determined at step S56 that the battery has not been replaced, i.e., the battery replacement flag is 0, the control unit 240 sets a value obtained by subtracting the value of the difference time included in the measurement result received at step S44 from the current date/time acquired at step S41, as the measurement date/time (step S57). Then, the process proceeds to step S55. FIG. 11 is a diagram schematically illustrating a configuration of data recorded in the measurement result recording unit 260, which corresponds to the example illustrated in FIG. 8A and FIG. 8B. In the example illustrated in FIG. 11, the battery replacement flags of the measurement results of the sequence numbers 28 to 30 are set to 0. As such, for the measurement result of the sequence number 30, 10:00:00 on July 5, which is a value obtained by subtracting 0 seconds, which is set as the difference time, from 10:00:00 on July 5, which is the current date/time, is set as the measurement date/time in accordance with the date/time computational equation of step S57. Likewise, for the measurement result of the sequence number 29, 9:36:00 on July 5, which is a value obtained by subtracting 1440 seconds from 10:00:00 on July 5, is set as the measurement date/time. Likewise, for the measurement result of the sequence number 28, 9:13:00 on July 5, which is a value obtained by subtracting 2820 seconds from 10:00:00 on July 5, is set as the measurement date/time.

When it is determined at step S56 that the battery has been replaced, i.e., the battery replacement flag is 1, the control unit 240 acquires the measurement result of one previous measurement date/time, i.e., a measurement result with a sequence number that is smaller by one from the measurement result recording unit 260 (step S58).

Then, the control unit 240 determines whether a valid value is set as the measurement date/time of the measurement result acquired at step S58 (step S59).

When it is determined to be No at step S59, i.e., when “invalid” is set as the measurement date/time included in the measurement result of one previous measurement date/time, the measurement date/time of the measurement result acquired at step S52 is also set as “invalid” (step S60). Then, the process proceeds to step S55.

When it is determined to be Yes at step S59, i.e., when a valid value is set as the measurement date/time included in the measurement result of one previous measurement date/time, a value obtained by adding the difference time included in the measurement result acquired at step S52 to one previous measurement date/time acquired at step S58 is set as the measurement date/time (step S61). Then, the process proceeds to step 55. In the example illustrated in FIG. 11, the battery replacement flag included in the measurement result of the sequence number 25 is 1, and one previous measurement date/time is 10:24:00 on July 3 of the sequence number 24. Accordingly, 10:48:00 on July 3, which is a value obtained by adding 1440 seconds, which is the difference time included in the measurement result of the sequence number 25 to 10:24:00 on July 3, is set as the measurement date/time. Likewise, for the measurement result of the sequence number 26, 11:12:00 on July 3, which is a value obtained by adding 1440 seconds to 10:48:00 on July 3, is set as the measurement date/time. Likewise, for the measurement result of the sequence number 27, 11:36:00 on July 3, which is a value obtained by adding 1440 seconds to 11:12:00 on July 3, is set as the measurement date/time.

For the measurement results received at step S43 to step S46, the control unit 240 repeats the processes of step S52 to step S55 up to the one with the largest sequence number (step S62).

In this manner, with the health information management system 1 according to the present embodiment, the measurement date/time information can be acquired at the smart phone 20 without providing a clock in the blood pressure monitor 10.

Exceptional Cases where Measurement Date/Time Cannot Be Specified

Through the above-described process, the date/time of the measurement of blood pressure information performed at the blood pressure monitor 10 provided with no clock can be specified at the smart phone 20, but in the following exceptional cases, the date/time of the measurement of the blood pressure information using the blood pressure monitor 10 cannot be specified at the smart phone 20.

Case 1

There is a case where the measurement result is not transmitted from the blood pressure monitor 10 to the smart phone 20 for some reason, or the measurement result transmitted from the blood pressure monitor 10 is not received by the smart phone 20 for some reason. In this case, in the smart phone 20, the measurement date/time cannot be specified because the measurement date/time as a reference is not held.

Case 2

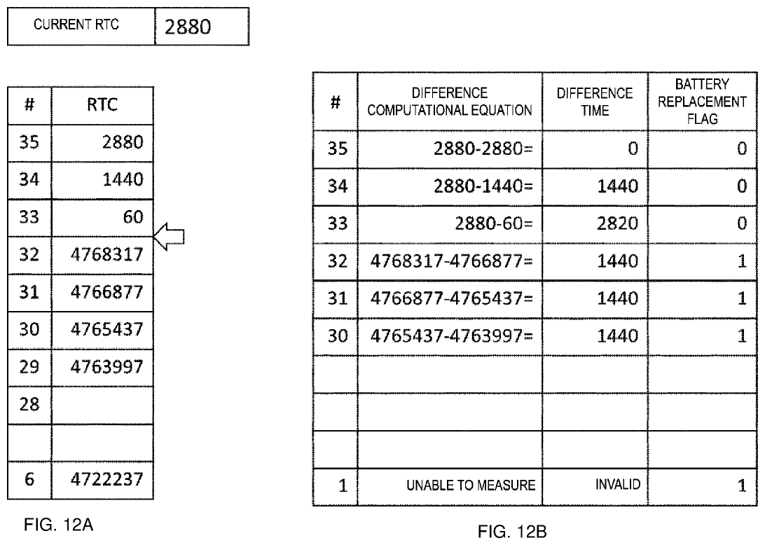

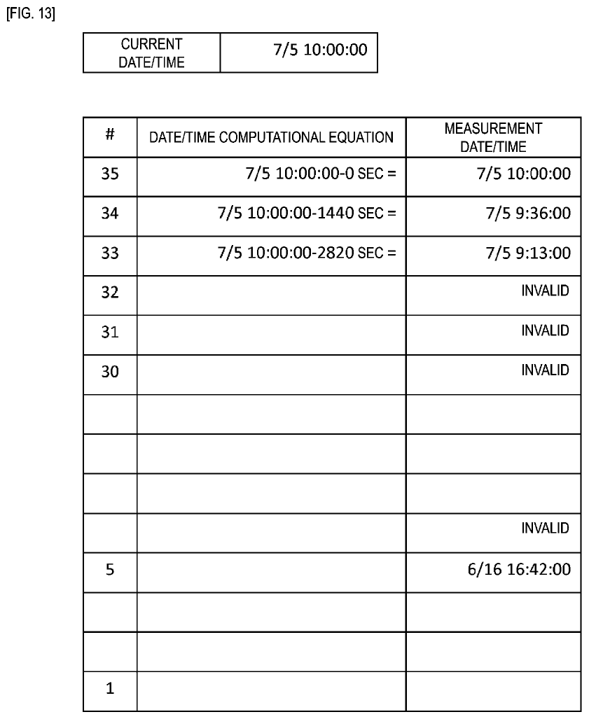

An exceptional case 2 will be described with reference to FIG. 12A, FIG. 12B and FIG. 13. FIG. 12A and FIG. 12B are diagrams schematically illustrating a configuration of data recorded in the measurement result recording unit 180 of the blood pressure monitor 10. In addition, FIG. 13 is a diagram schematically illustrating a configuration of data of the measurement result recording unit 260 of the smart phone 20.

Here, the number of cases N that can be recorded in the measurement result recording unit 180 of the blood pressure monitor 10 is limited, and when measurements are made more than N cases, the measurement result is erased in the order from the oldest measurement result. The case 2 is a case where the measurement has been made more than N times after the smart phone 20 has finally received the measurement result, and the measurement result is transmitted from the blood pressure monitor 10 to the smart phone 20 after the battery has been replaced between measurements within the most recent N times.

In the example illustrated in FIG. 12A, N=30 holds, five cases of the sequence numbers 1 to 5 are erased, and the sequence numbers 6 to 35 are recorded. The measurement results of the sequence numbers 1 to 5 have already been transmitted from the blood pressure monitor 10 to the smart phone 20, and the measurement date/time is specified as 16:42:00 on June 16 for the measurement result of the sequence number 5 as illustrated in FIG. 13. The measurement results of the sequence number 5 and before have already been erased in the blood pressure monitor 10, and thus the difference time, for the measurement result of the sequence number 6, cannot be calculated in accordance with the difference computational equation, and is set as “invalid”. As a result, at the smart phone 20, for the measurement results of the sequence numbers 6 to 32, there is no measurement date/time as a reference, and therefore the measurement date/time cannot be specified, and, each measurement date/time is set as “invalid”.

In the above-described process, for the measurement result set as “invalid” as the measurement date/time, i.e., the measurement results of the sequence numbers 6 to 32 in the example illustrated in FIG. 13, the data of the difference time received from the blood pressure monitor 10 may be stored in the measurement result recording unit 260. In this manner, even for the data set as “invalid” as the measurement date/time, the interval of the measurement dates/times can be specified, and thus a relative change with time of blood pressure information can be displayed on the touch panel display 220.

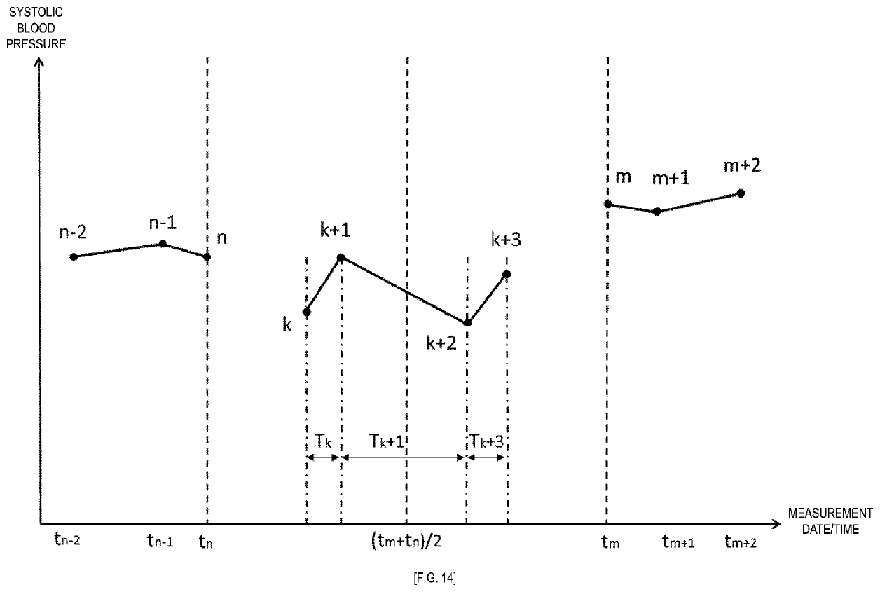

FIG. 14 illustrates an example of a display of a change with time of a systolic blood pressure as blood pressure information.

Assume here that for measurement results of the sequence number n or smaller and the sequence number m or greater, measurement dates/times of tₙ, tₘ and the like are specified. On the other hand, the measurement date/time of each of measurement results of sequence numbers k to k+3 is set as “invalid”. Here, regarding the measurement results of sequence numbers k to k+3, the intervals Tₖ, Tₖ₊₁ and Tₖ₊₂ of the measurement dates/times of the sequence numbers k to k+1, the sequence numbers k+1 to k+2 and the sequence numbers k+2 to k+3, respectively, are specified by the count values of the RTC. Therefore, the intervals in the time direction of the data corresponding to these sequence numbers are set to correspond to the intervals of the respective count values of the RTC. In the case where a temporary graph of a measurement result whose measurement date/time is “invalid” is disposed in a graph representing a time-series change of measurement results, it can be disposed such that the center in the time direction of the measurement result whose measurement date/time is “invalid” corresponds to the center value of valid measurement dates/times before and after it. A temporary graph of the measurement results of the data of the sequence numbers k to k+3 is disposed in a time-series graph of the measurement results of the systolic blood pressure such that the center of their measurement time interval corresponds to the center value, here (tₘ+tₙ)/2, of the measurement dates/times of a measurement result (sequence number n) that is newest among the measurement results whose measurement date/time is specified and older than the oldest measurement result whose measurement date/time is “invalid”, and a measurement result (sequence number m) that is oldest among the measurement results whose measurement date/time is specified and newer than the newest measurement result whose measurement date/time is “invalid”.

In this manner, even for a measurement result whose exact measurement date/time cannot be specified, it is possible to know a relative time-series change with other measurement results whose measurement date/time can be specified, thus providing useful information for health management.

It is to be noted that in the example illustrated in FIG. 14, the sequence number n and the sequence number k, and the sequence number k+3 and the sequence number m are not necessarily be continuous, but they may be continuous.

Case 3

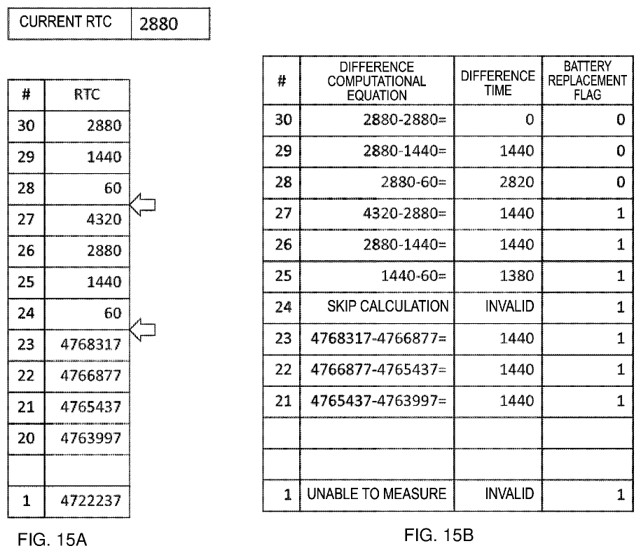

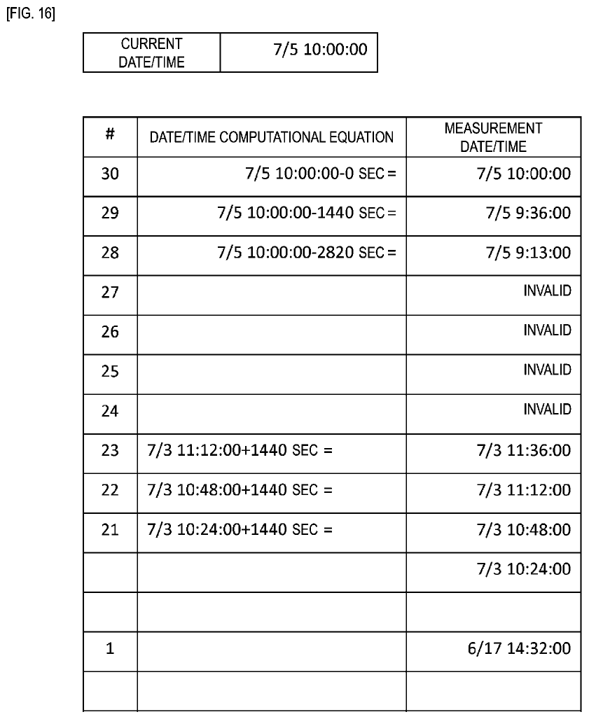

An exceptional case 3 will be described below with reference to FIG. 15A, FIG. 15B and FIG. 16. FIG. 15A and FIG. 15B are diagrams schematically illustrating a configuration of data recorded in the measurement result recording unit 180 of the blood pressure monitor 10. In addition, FIG. 16 is a diagram schematically illustrating a configuration of data of the measurement result recording unit 260 of the smart phone 20.

The case 3 is a case where the battery is replaced two or more times after the last transmission of a measurement result from the blood pressure monitor 10 to the smart phone 20. In such a case, while the measurement dates/times can be specified for the measurement results up to the replacement of the first battery, the measurement dates/times cannot be specified for the measurement results up to the latest battery replacement after the second battery replacement.

In the example illustrated in FIG. 15A, the battery has been replaced two times, between the sequence number 23 and sequence number 24, and between the sequence number 27 and sequence number 28. For the sequence numbers 21 to 23, the sequence numbers 25 to 27, and the sequence numbers 28 to 30, the difference time can be calculated in accordance with the difference computational equation described in the first embodiment, but for the measurement result of the sequence number 24, “invalid” is set as a difference time because the difference calculation cannot be made. At this time, the measurement results of the sequence numbers 1 to 20 have already been transmitted from the blood pressure monitor 10 to the smart phone 20, and the specified measurement dates/times are recorded in the measurement result recording unit 260 as illustrated in FIG. 16. Therefore, the measurement dates/times of the measurement results of the sequence numbers 21 to 23 and the sequence numbers 28 to 30 can be specified in accordance with the date/time computational equation described in the first embodiment. However, for the measurement results of the sequence numbers 24 to 27, there is no measurement date/time as a reference, and the measurement dates/times cannot be specified, and as such, each measurement date/time is set as “invalid”.

### Second Embodiment

Next, a second embodiment of the present invention will be described. The hardware configuration of the health information management system 1 according to the present embodiment is the same as that of the first embodiment. In addition, the measurement of the blood pressure information using the blood pressure monitor 10, and the entire flow of the processes from the measurement of blood pressure information at the blood pressure monitor to the process of transmitting measurement results to the smart phone 20 are also the same as those of the first embodiment. The same processes as those of the first embodiment are denoted with the same reference numerals and the description thereof is omitted.

In the present embodiment, the transmission of measurement results from the blood pressure monitor 10 to the smart phone 20, and the details of the measurement date/time specifying process at the smart phone 20 having received measurement results from the blood pressure monitor 10 are different from those of the first embodiment, and therefore these processes will be described below.

Transmission of Measurement Result from Blood Pressure Monitor to Smart Phone

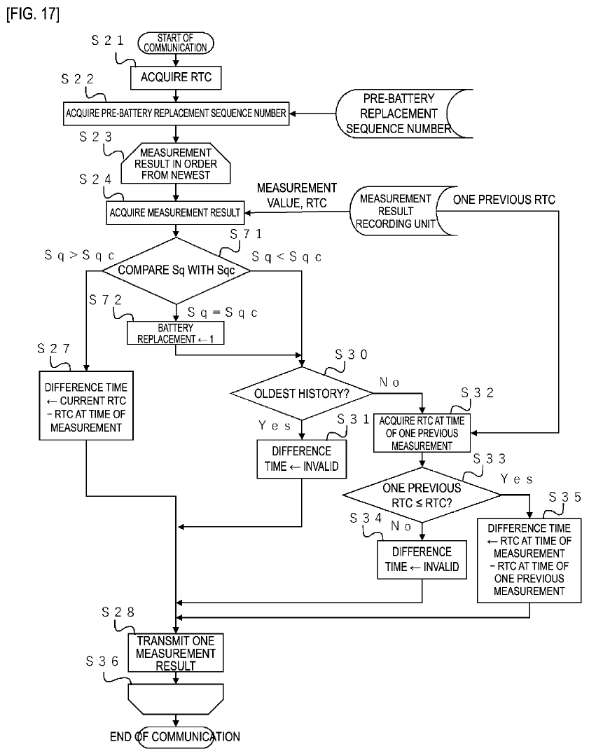

FIG. 17 is a flowchart illustrating a procedure of a process of transmitting data including a measurement result from the blood pressure monitor 10 to the smart phone 20 according to the second embodiment. The processes common to the first embodiment are denoted with the same reference numerals and the description thereof is omitted.

In the present embodiment, instead of assigning battery replacement flags to all measurement results stored in the measurement result memory and transmitting them to the smart phone 20, only the measurement result to which the same sequence number as the pre-battery replacement sequence number is assigned is assigned with a battery replacement flag 1 and transmitted.

As illustrated in FIG. 17, when acquiring a measurement result from the measurement result recording unit 180, the control unit 150 compares the sequence number of the measurement result (Sq) and the pre-battery replacement sequence number (Sqc) acquired at step S22 (step S71).

At step S71, when it is determined that Sq>Sqc holds, i.e., when the sequence number of the measurement result is determined to be greater than the pre-battery replacement sequence number, it is the measurement result after the battery replacement, and therefore the process proceeds to step S27.

At step S71, when it is determined that Sq=Sqc holds, i.e., when the sequence number of the measurement result is determined to be the same as the pre-battery replacement sequence number, the battery replacement flag 1 is assigned (step S72), and the process proceeds to step S30.

At step S71, when it is determined that Sq<Sqc holds, i.e., when the sequence number of the measurement result is determined to be smaller than the pre-battery replacement sequence number, it is the pre-battery replacement measurement result, and therefore the process proceeds to step

S30.

The processes at and after step S27 and step S30 are the same as those of the first embodiment, and therefore the description thereof is omitted. At this time, in the measurement results transmitted to the smart phone 20 at step S28, only measurement results with the same sequence number as the pre-battery replacement sequence number include the battery replacement flag 1, and the other measurement results do not include the battery replacement flag.

Measurement Date/Time Specifying Process at Smart Phone Having Received Measurement Results from Blood Pressure Monitor

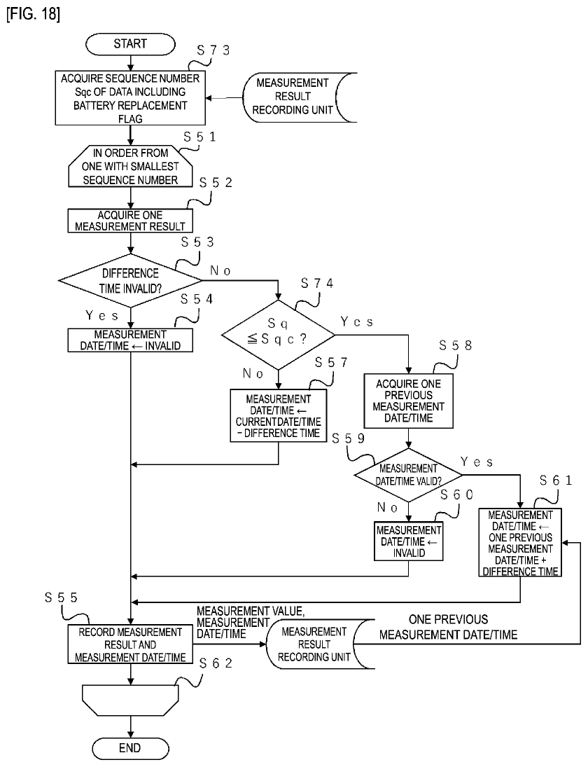

FIG. 18 is a flowchart illustrating a procedure of a process of specifying the measurement date/time at the smart phone 20 having received data including a measurement result from the blood pressure monitor 10 according to the second embodiment. The processes common to the first embodiment are denoted with the same reference numerals and the description thereof is omitted. The process of receiving data including a measurement result from the blood pressure monitor 10 prior to the process of specifying the measurement date/time that is executed at the smart phone 20 illustrated in FIG. 18 is common to the first embodiment illustrated in FIG. 9. It should be noted that in the present embodiment, when data including a battery replacement flag is received together with the measurement result, the sequence number of the data, i.e., Sqc, is stored in a predetermined region of the measurement result recording unit 260.

In the present embodiment, in the measurement results received from the blood pressure monitor 10, measurement results including the battery replacement flag are measurement results to which the same sequence number as the pre-battery replacement sequence number is assigned. Therefore, in the measurement results received in descending order of the sequence number, the measurement results before reception of the battery replacement flag are determined to be those after the battery replacement, and the measurement results after the reception of the battery replacement flag are determined to be those before the battery replacement, and thus, the measurement date/time is specified in accordance with the determination results.

First, the sequence number Sqc of data including a battery replacement flag is acquired from the predetermined region of the measurement result recording unit 260 (step S73).

The processes of steps S51 to S55 are the same as those of the first embodiment.

When it is determined at step S53 that the difference time is not invalid, the control unit 240 determines the sequence number Sq of the measurement result acquired at step S52 is not greater than Sqc (step S74).

When No at step S74, i.e., when the sequence number Sq is greater than Sqc, the measurement result is the measurement result after the battery replacement, and therefore the process proceeds to step S57.

When Yes at step S74, i.e., when the sequence number Sq is not greater than Sqc, the measurement result is the measurement result before battery replacement, and therefore the process proceeds to step S58.

The processes at and after step S54, step S57 and step S58 are the same as those of the first embodiment, and therefore the description thereof is omitted.

In this manner, when measurement results are transmitted from the blood pressure monitor 10, only the measurement result to which the same sequence number as the pre-battery replacement sequence number is assigned is assigned with the battery replacement flag 1 and transmitted to the smart phone 20, and thus the processing load at the time of transmission of measurement results can be reduced, and the time required for the process can be shortened. In addition, the transmission time can also be shortened because the amount of data to be transmitted is reduced.

### Third Embodiment

Next, a third embodiment of the present invention will be described. The hardware configuration of the health information management system 1 according to the present embodiment is the same as that of the first embodiment. In addition, the measurement of the blood pressure information using the blood pressure monitor 10, and the entire flow of the processes from the measurement of blood pressure information at the blood pressure monitor to the process of transmitting measurement results to the smart phone 20 are also the same as those of the first embodiment. The same processes as those of the first embodiment are denoted with the same reference numerals and the description thereof is omitted.

In the present embodiment, the transmission of measurement results from the blood pressure monitor 10 to the smart phone 20, and the details of the measurement date/time specifying process at the smart phone 20 having received measurement results from the blood pressure monitor 10 are different from those of the first embodiment, and therefore these processes will be described below.

Transmission of Measurement Result from Blood Pressure Monitor to Smart Phone

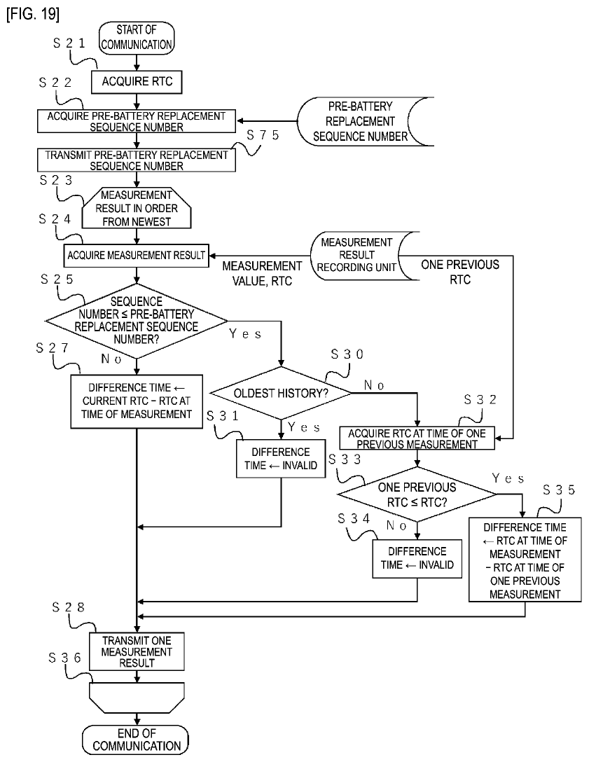

FIG. 19 is a flowchart illustrating a procedure of a process of transmitting data including a measurement result from the blood pressure monitor 10 to the smart phone 20 according to the third embodiment. The processes common to the first embodiment are denoted with the same reference numerals and the description thereof is omitted.

In the present embodiment, separately from the measurement result stored in the measurement result memory, the pre-battery replacement sequence number is transmitted from the blood pressure monitor 10 to the smart phone 20.

As illustrated in FIG. 19, the control unit 150 acquires a pre-battery replacement sequence number stored in a predetermined region of the measurement result recording unit 180 (step S22), and transmits it to the smart phone 20 through the communication unit 130 (step S75). The processes at and after step S23 are the same as those of the first embodiment, and therefore the description thereof is omitted.

Measurement Date/Time Specifying Process at Smart Phone Having Received Measurement Results from Blood Pressure Monitor

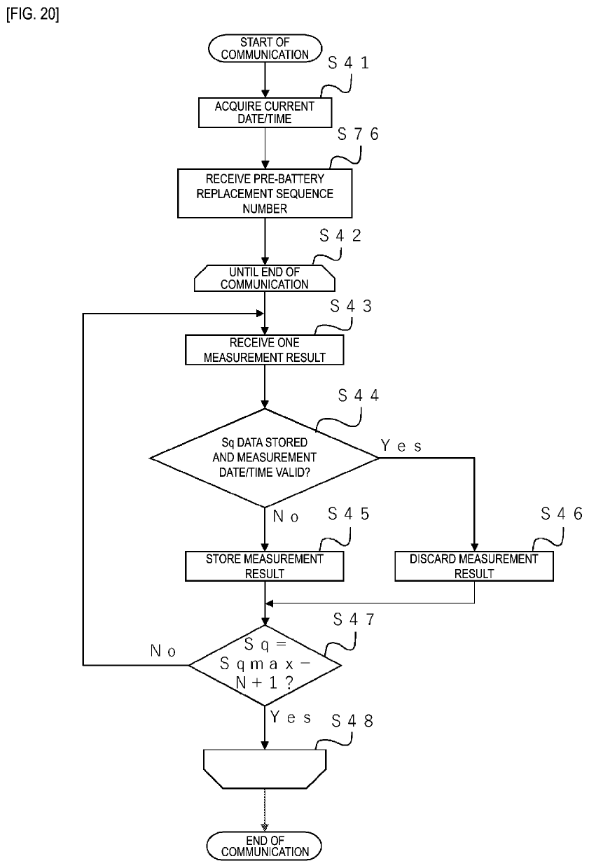

FIG. 20 is a flowchart illustrating a procedure of a process of receiving data including a measurement result from the blood pressure monitor 10 at the smart phone 20 according to the third embodiment. The processes common to the first embodiment are denoted with the same reference numerals and the description thereof is omitted.

After the control unit 240 has acquired the current date/time at step S41, the pre-battery replacement sequence number is received from the blood pressure monitor 10 (step S76).

The processes at and after step S42 are common to the first embodiment illustrated in FIG. 9.

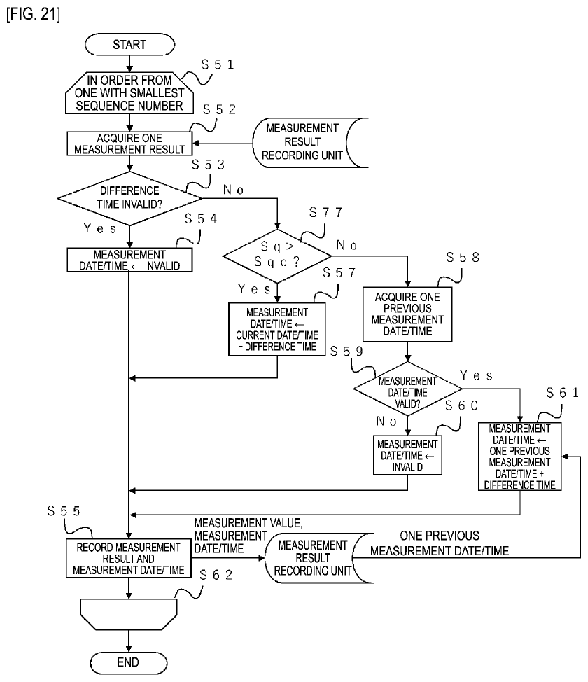

FIG. 21 is a flowchart illustrating a procedure of a process of specifying the measurement date/time to be executed at the smart phone 20 for the measurement result received in accordance with FIG. 20. A measurement date/time specifying process at the control unit 240 will be described below with reference to FIG. 21.

In the present embodiment, whether the sequence number assigned to the measurement result received from the blood pressure monitor 10 is greater than the pre-battery replacement sequence number is determined. Then, the measurement result to which a sequence number greater than the pre-battery replacement sequence number is assigned is determined to be a measurement result after the battery replacement, and the measurement result to which a sequence number not greater than the pre-battery replacement sequence number is assigned is determined to be a measurement result before battery replacement, and thus, the measurement date/time is specified in accordance with the determination results.

The control unit 240 repeats a process in the order from the one with the smallest sequence number for the measurement results received at step S43 to step S46 (step S51).

First, the control unit 240 acquires one measurement result with the smallest sequence number among the measurement results received at step S43 to step S46 from the measurement result recording unit 260 (step S52), and determines whether the difference time is invalid (step S53).

When it is determined at step S53 that the difference time is determined to be not invalid, the control unit 240 determines whether the sequence number (Sq) assigned to the measurement result is greater than the pre-battery replacement sequence number (Sqc) (step S77).

When it is determined at step S77 that the sequence number (Sq) assigned to the measurement result is greater than the pre-battery replacement sequence number (Sqc), the process proceeds to step S57. When it is determined at step S77 that the sequence number (Sq) assigned to the measurement result is not greater than the pre-battery replacement sequence number (Sqc), the process proceeds to step S58. The processes at and after step S54, step S57 and step S58 are the same as those of the first embodiment, and therefore the description thereof is omitted.

In this manner, when measurement results are transmitted from the blood pressure monitor 10, the pre-battery replacement sequence number is transmitted to the smart phone 20 separately from the measurement result, and thus, in comparison with the case where each measurement result is assigned with the battery replacement flag and transmitted, the processing load at the time of transmission of measurement results can be reduced, and the time required for the process can be shortened. In addition, the transmission time can also be shortened because the amount of data to be transmitted is reduced.

### Fourth Embodiment

Next, a fourth embodiment of the present invention will be described. The hardware configuration of the health information management system 1 according to the present embodiment is the same as that of the first embodiment. In addition, the measurement of the blood pressure information using the blood pressure monitor 10, and the entire flow of the processes from the measurement of blood pressure information at the blood pressure monitor to the process of transmitting measurement results to the smart phone 20 are also the same as those of the first embodiment. The same processes as those of the first embodiment are denoted with the same reference numerals and the description thereof is omitted.

In the present embodiment, the transmission of measurement results from the blood pressure monitor 10 to the smart phone 20, and the details of the measurement date/time specifying process at the smart phone 20 having received measurement results from the blood pressure monitor 10 are different from those of the first embodiment. In the first embodiment to third embodiment, the difference time is calculated at the blood pressure monitor 10 from the RTC at the time of measurement and the like, and is transmitted to the smart phone 20. Then, the measurement date/time is specified at the smart phone 20 on the basis of the difference time, the battery replacement flag and the like. In the present embodiment, the RTC at the time of measurement and the like are transmitted to the smart phone 20 without calculating the difference time at the blood pressure monitor 10, and the measurement date/time is specified at the smart phone 20 on the basis of the difference time and the like. It is to be noted that also for the normal processes described in the first embodiment, the count value of the RTC at the time of measurement may be transmitted from the blood pressure monitor 10 to the smart phone 20 together with the measurement value as in the present embodiment. In this case, the count value of the RTC as of the transmission time is acquired at step S4 in FIG. 5, and is thereafter transmitted to the smart phone 20. Then, on the smart phone 20 side, the measurement date/time can be calculated by calculating the difference between the count value of the RTC as of the transmission time and the count value of the RTC at the time of measurement, and subtracting the difference of the count value of the RTC from the date/time information as of the transmission time.

Transmission of Measurement Result from Blood Pressure Monitor to Smart Phone

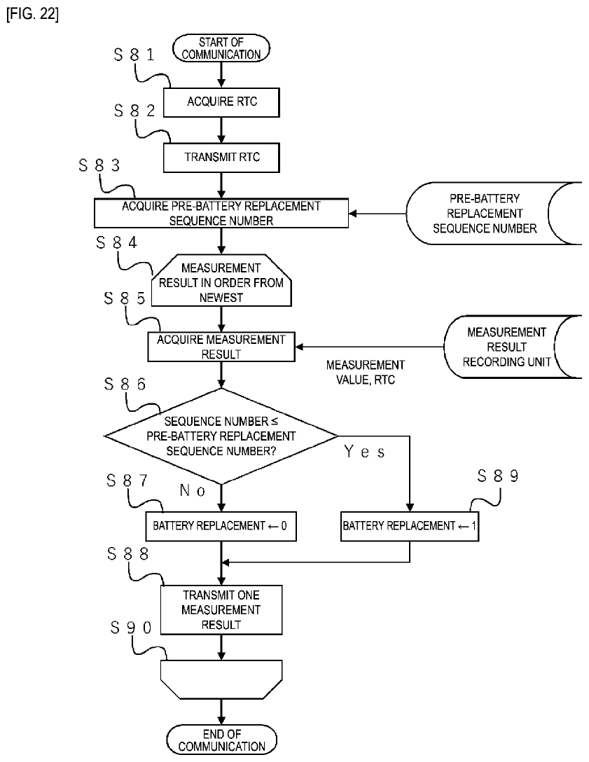

FIG. 22 is a flowchart illustrating a procedure of a process of transmitting data including a measurement result from the blood pressure monitor 10 to the smart phone 20 according to the fourth embodiment.

First, the control unit 150 establishes communication with the smart phone 20 through the communication unit 130. When the communication between the blood pressure monitor 10 and the smart phone 20 is established, the control unit 150 acquires the count value of the RTC as of the transmission time (step S81).

Next, the control unit 150 transmits the acquired count value of the RTC as of the transmission time to the smart phone 20 through the communication unit 130 (step S82).

Next, the control unit 150 acquires a pre-battery replacement sequence number from a predetermined region of the measurement result recording unit 180 (step S83).

Next, in order from the newest measurement result (step S84), i.e., in descending order from the one with the largest sequence number, the control unit 150 acquires blood pressure information and the count value of the RTC at the time of measurement as a measurement result from the measurement result recording unit 180 (step S85).

Then, the control unit 150 determines whether the sequence number of the measurement result acquired at step S85 is not greater than the pre-battery replacement sequence number acquired at step S83 (step S86).

When it is determined to be No at step S86, i.e., when the sequence number of the measurement result acquired at step S85 is greater than the pre-battery replacement sequence number acquired at step S83, the control unit 150 sets the battery replacement flag to 0 because it is the measurement result after the battery replacement (step S87).

Then, the control unit 150 transmits the blood pressure information, the count value of the RTC at the time of measurement, and the battery replacement flag as the measurement result for one case to the smart phone 20 through the communication unit 130 (step S88).

When it is determined to be Yes at step S86, i.e., when the sequence number of the measurement result acquired at step S85 is not greater than the pre-battery replacement sequence number acquired at step S83, the control unit 150 sets the battery replacement flag to 1 because it is the pre-battery replacement measurement result (step S89). Then, the process proceeds to step S88.

After the processes from step S85 to step S88 are repeated for all measurement results in descending order from the newest measurement result (step S90), the control unit 150 disconnects and terminates the communication with the smart phone 20 through the communication unit 130.

Measurement Date/Time Specifying Process at Smart Phone Having Received Measurement Results from Blood Pressure Monitor

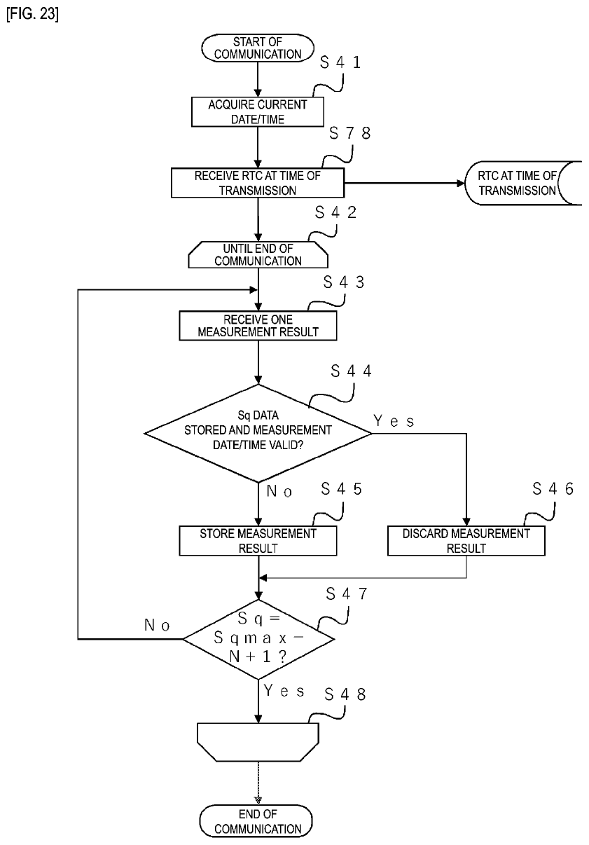

FIG. 23 is a flowchart illustrating a procedure of a process of receiving data including a measurement result from the blood pressure monitor 10 at the smart phone 20 according to the fourth embodiment. The processes common to the first embodiment are denoted with the same reference numerals and the description thereof is omitted.

When the communication with the blood pressure monitor 10 is established and started, the control unit 240 acquires the current date/time information from the clock unit 250 (step S41).

Next, the control unit 240 receives the count value of the RTC at the time of transmission from the blood pressure monitor 10 through the communication unit 210 (step S78). The received count value of the RTC at the time of transmission is stored in a predetermined region of the nonvolatile memory included in the storage unit 230.

The processes at and after step S42 are common to the first embodiment illustrated in FIG. 9.

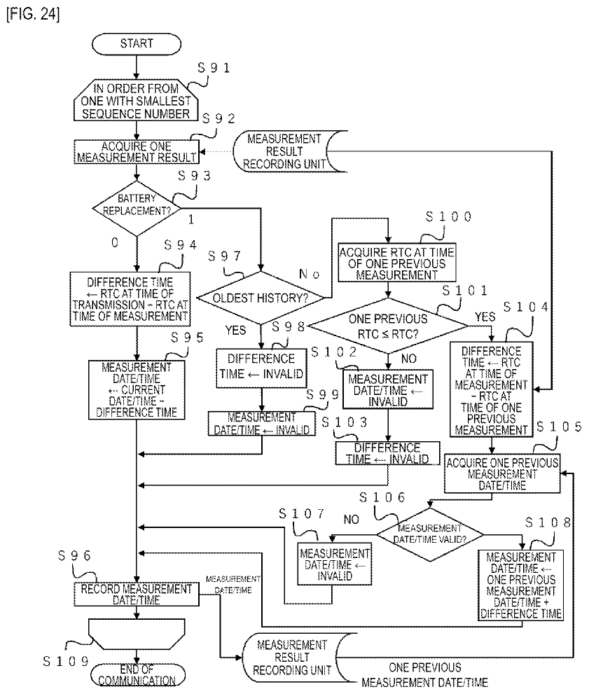

FIG. 24 is a flowchart illustrating a procedure of a process of specifying the measurement date/time to be executed at the smart phone 20 for the measurement result received in accordance with FIG. 23. A measurement date/time specifying process at the control unit 240 will be described below with reference to FIG. 24.

The control unit 240 repeats the process on the measurement results received at step S43 to step S46 in the order from the one with the smallest sequence number (step S91).

Next, the control unit 240 acquires the measurement result one by one from the measurement result recording unit 260 (step S92).

Next, the control unit 240 determines whether the value of the battery replacement flag included in the measurement result acquired at step S92 is 0 or 1 (step S93).

Specifically, the case where the value of the battery replacement flag is determined to be 0 at step S93 is a case where the measurement result acquired at step S92 is a post-battery replacement result. In this case, as the difference time, the control unit 240 sets a value obtained by subtracting the count value of the RTC at the time of measurement of the measurement result acquired at step S92 from the count value of the RTC at the time of transmission received at step S78 (step S94).

Next, as the measurement date/time, the control unit 240 sets a value obtained by subtracting the value of the difference time calculated at step S94 from the current date/time acquired at step S41 (step S95). Then, the measurement date/time set in this manner is recorded in the measurement result recording unit 260 (step S96).

When it is determined at step S93 that the value of the battery replacement flag is 1, i.e., when the measurement result acquired at step S92 is a pre-battery replacement measurement result, the control unit 240 determines whether the measurement result is the oldest history (step S97). Here, whether it is the oldest history is determined based on whether a measurement result with a smaller sequence number is received.

When it is determined to be Yes at step S97, the control unit 240 sets “invalid” as the difference time (step S98), and sets “invalid” as the measurement date/time (step S99). Then, the process proceeds to step S96.

When it is determined to be No at step S97, the control unit 240 acquires the count value of the RTC at the time of one previous measurement (step S100).

Then, the control unit 240 determines whether the count value of the RTC at the time of one previous measurement acquired at step S100 is not greater than the count value of the RTC received at step S92 (step 101).

When it is determined to be No at step S101, the control unit 240 sets “invalid” as the difference time (step S102), and sets “invalid” as the measurement date/time (step S103). Then, the process proceeds to step S96.

When it is determined to be Yes at step S101, the control unit 240 sets, as the difference time, a value obtained by subtracting the count value of the RTC at the time of one previous measurement acquired at step S100 from the count value of the RTC at the time of measurement received at step S92 (step S104). Then, the one previous measurement date/time is acquired (step S105).

Next, the control unit 240 determines whether a valid value is set as the measurement date/time of the measurement result acquired at step S105 (step S106).

When it is determined to be No at step S106, i.e., when “invalid” is set as the measurement date/time included in the measurement result of one previous measurement date/time, the measurement date/time of the measurement result acquired at step S92 is also set as “invalid” (step S107). Then, the process proceeds to step S96.

When it is determined to be Yes at step S106, i.e., when a valid value is set as the measurement date/time included in the measurement result of one previous measurement date/time, a value obtained by adding the difference time calculated at step S104 to one previous measurement date/time acquired at step S105 is set as the measurement date/time (step S108). Then, the process proceeds to step S96.

The control unit 240 repeats the processes of step S93 to step S96 for the measurement results received at step S43 to step S46 up to the one with the largest sequence number (step S109).

In this manner, with the health information management system 1 according to the present embodiment, the measurement date/time information can be acquired at the smart phone 20 without providing a clock in the blood pressure monitor 10.

In addition, in this manner, when measurement results are transmitted from the blood pressure monitor 10 to the smart phone 20, the count value of the RTC at the time of measurement is directly transmitted to the smart phone 20 side and the required difference time is calculated on the smart phone 20 side instead of calculating the difference time on the blood pressure monitor 10 side. Thus, the load of the process of the blood pressure monitor 10 at the time of transmission can be reduced, and the time required for the process can be shortened.

In the fourth embodiment, at the blood pressure monitor 10, all measurement results to which the sequence number smaller than the pre-battery replacement sequence is assigned are assigned with the battery replacement flag 1 and transmitted to the smart phone 20, but only the measurement result to which the same sequence number as the pre-battery replacement sequence number is assigned may be assigned with the battery replacement flag 1 and transmitted to the smart phone 20 as in the second embodiment. In addition, the pre-battery replacement sequence number may be transmitted from the blood pressure monitor 10 to the smart phone 20 separately from the measurement result stored in the measurement result memory as in the third embodiment.

### Fifth Embodiment

Next, a fifth embodiment of the present invention will be described. The hardware configuration of the health information management system 1 according to the present embodiment is the same as that of the first embodiment. In the present embodiment, as in the fourth embodiment, the count value of the RTC at the time of the blood pressure information measurement is transmitted from the blood pressure monitor 10 to the smart phone 20, and the calculation of the difference of the count value of the RTC is performed on the smart phone 20 side. The same processes as those of the first embodiment and the fourth embodiment are denoted with the same reference numerals and the description thereof is omitted.

In the present embodiment, as battery replacement history information related to a battery replacement history of the blood pressure monitor 10, battery replacement count information representing the number of times of battery replacement up to the current time is held in a predetermined region in the nonvolatile memory included in the storage unit 170. The initial value of the battery replacement count information is 0, and is counted up by one at the first power-on after the battery replacement.

Measurement of Blood Pressure Information Using Blood Pressure Monitor

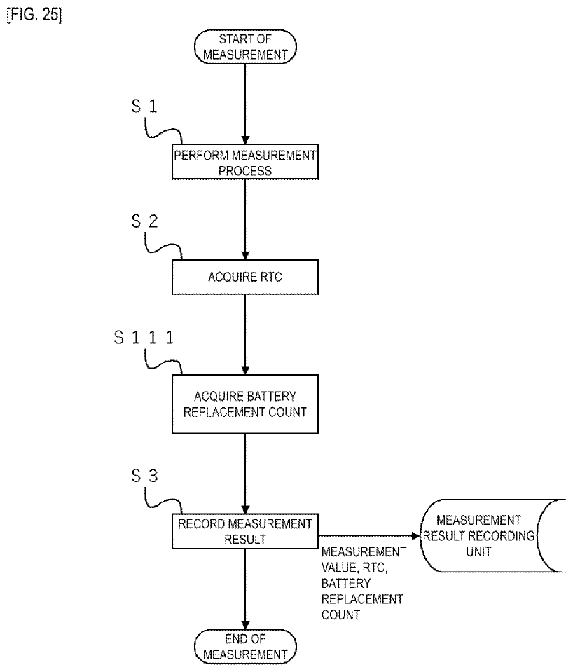

FIG. 25 is a flowchart illustrating a processing procedure of measurement of the blood pressure information and recording of measurement results at the blood pressure monitor 10 according to the present embodiment. The measurement process (step S1) and the acquisition of the count value of the RTC (step S2) are the same as those of the first embodiment. In the present embodiment, the control unit 150 acquires the battery replacement count information at the time of the blood pressure information measurement from the predetermined region of the storage unit 170 (step S111). Then, when recording the measurement result (step S3), the control unit 150 stores, in the measurement result recording unit 180, the battery replacement count information in addition to the measurement value of the blood pressure information and the count value of the RTC.

Transmission of Measurement Results to Smart Phone

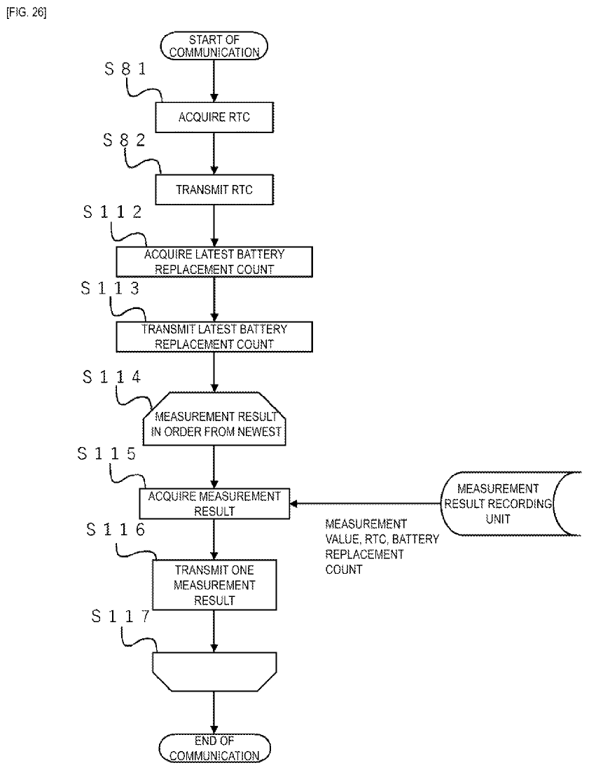

FIG. 26 is a flowchart illustrating a flow of a process of transmitting measurement results from the blood pressure monitor 10 to the smart phone 20 according to the present embodiment.

The control unit 150 acquires the count value of the RTC as of the transmission time after the establishment of the communication with the smart phone 20 (step S81), and transmits it to the smart phone 20 through the communication unit 130 (step S82). In the present embodiment, next, the control unit 150 acquires the latest battery replacement count information from the predetermined region of the storage unit 170 (step S112), and transmits it to the smart phone 20 through the communication unit 130 (step S113).

Next, in order from the newest measurement result (step S114), i.e., in descending order from the one with the largest sequence number, the control unit 150 acquires the blood pressure information, the count value of the RTC at the time of measurement and the battery replacement count information at the time of measurement as a measurement result from the measurement result recording unit 180 (step S115). Then, the control unit 150 transmits the blood pressure information, the count value of the RTC at the time of measurement and the battery replacement count information at the time of measurement as the measurement result for one case to the smart phone 20 through the communication unit 130 (step S116).

After repeating the processes of step S115 and step S116 for all measurement results in descending order from the newest measurement result (step S117), the control unit 150 disconnects and terminates the communication with the smart phone 20 through the communication unit 130.

Measurement Date/Time Specifying Process at Smart Phone Having Received Measurement Results from Blood Pressure Monitor

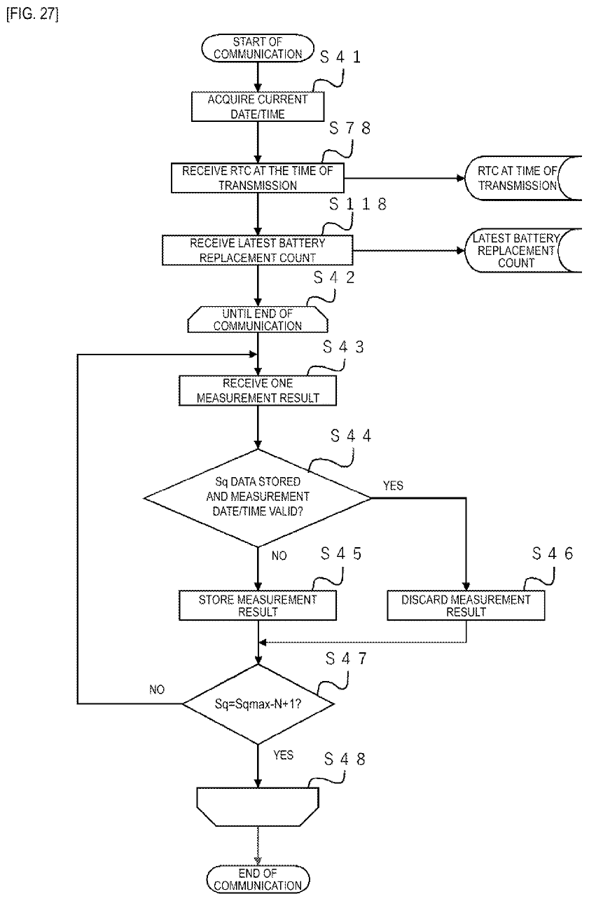

FIG. 27 is a flowchart illustrating a procedure of a process of receiving data including a measurement result from the blood pressure monitor 10 at the smart phone 20.

When the communication with the blood pressure monitor 10 is established and started, the control unit 240 acquires the current date/time information from the clock unit 250 (step S41).

Next, the control unit 240 receives the count value of the RTC at the time of transmission from the blood pressure monitor 10 through the communication unit 210 (step S77). The received count value of the RTC at the time of transmission is stored in a predetermined region of the nonvolatile memory included in the storage unit 230.

Then, the control unit 240 receives the latest battery replacement count information from the blood pressure monitor 10 through the communication unit 210 (step S118). The received latest battery replacement count information is stored in the predetermined region of the nonvolatile memory included in the storage unit 230.

The processes at and after step S42 are common to the first embodiment illustrated in FIG. 9.

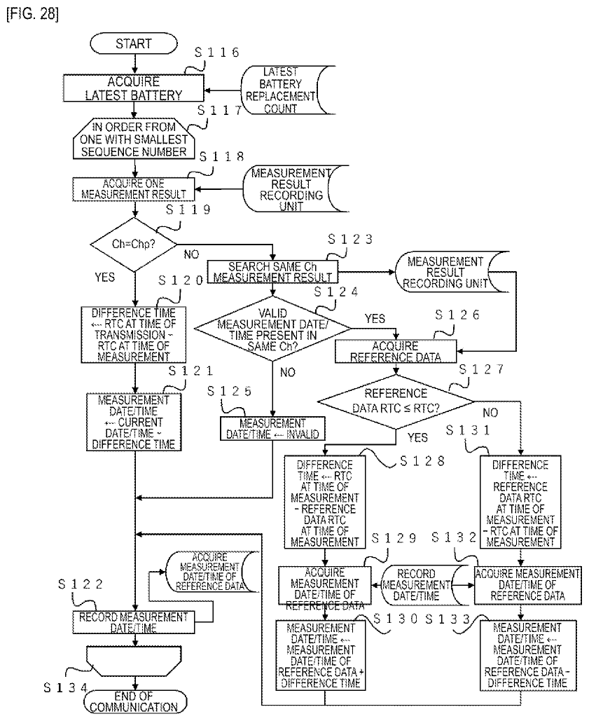

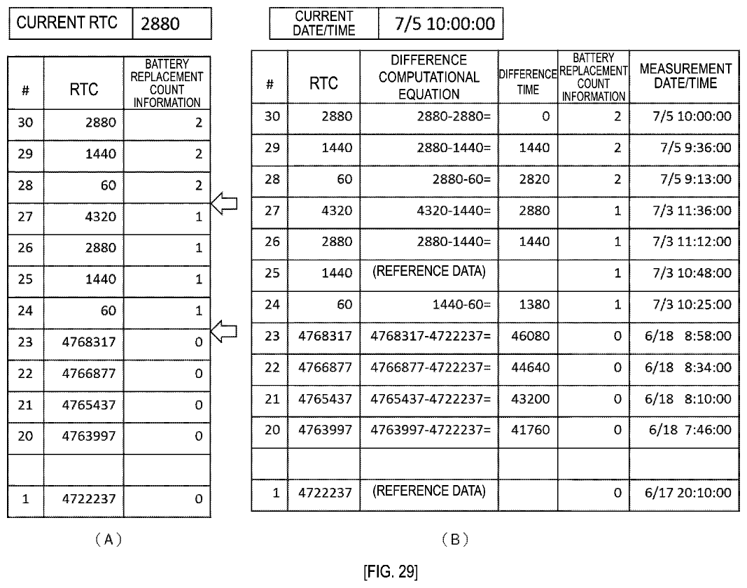

FIG. 28 is a flowchart illustrating a procedure of a process of specifying the measurement date/time to be executed at the smart phone 20 for the measurement result received in accordance with FIG. 27. FIG. 29A is a diagram illustrating an example of a data configuration of the measurement result recording unit 180 in the blood pressure monitor 10 according to the present embodiment. FIG. 29B is a diagram illustrating a detailed example of a process of specifying a measurement date/time and an example of a part of a data configuration in the smart phone 20 according to the present embodiment. A measurement date/time specifying process at the control unit 240 will be described below with reference to FIG. 28. In the example illustrated in FIG. 29A, the number of cases that can be recorded in the measurement result recording unit 180 of the blood pressure monitor 10 is 30. In this example, the first battery replacement is performed between the measurement of the sequence number 23 and the measurement of the sequence number 24, and the second battery replacement is performed between the measurement of the sequence number 27 and the measurement of sequence number 28. Accordingly, 0 is recorded as the battery replacement count information for the sequence numbers 1 to 23, 1 is recorded as the battery replacement count information for the sequence numbers 24 to 27, and 2 is recorded as the battery replacement count information for the sequence numbers28 to 30. Here, the latest battery replacement count is 2. In addition, in FIG. 29B, the battery replacement count information and the count value of the RTC of the measurement result are also recorded in addition to the blood pressure information. In the example illustrated in FIG. 29B, the measurement results of the sequence number 1 and the sequence number 25 at the time of measurement are appropriately transmitted to the smart phone 20, and valid measurement dates/times are specified.

The control unit 240 acquires the latest battery replacement count information stored in the predetermined region of the storage unit 230 (step S116). In the example illustrated in FIG. 29A, “2” is acquired.

Next, the control unit 240 repeats the process on the measurement results received at step S43 to step S46 in the order from the one with the smallest sequence number (step S117).

First, the control unit 240 acquires the measurement result one by one from the measurement result recording unit 260 (step S118).

Then, the control unit 240 determines whether the battery replacement count information (Ch) included in the measurement result acquired at step S118 is equal to the latest battery replacement count information (Chp) received at step S116 (step S119). Here, as described above, Chp=2 holds.

A case where it is determined at step S119 that Ch=Chp holds is a case where no battery replacement is performed after the measurement of the measurement result acquired at step S118. In this case, as the difference time, the control unit 240 sets a value obtained by subtracting the count value of the RTC at the time of measurement of the measurement result acquired at step S118 from the count value of the RTC at the time of transmission received at step S78 (step S120). In the example illustrated in FIG. 29A, for the measurement results of the sequence numbers 28 to 30, difference times 0, 1440 and 2820 are obtained as a result of subtraction of count values of 2880, 1440 and 60 of the RTC at the time of measurement of the measurement results from the count value of 2880 of the RTC at the time of transmission. Next, as the measurement date/time, the control unit 240 sets a value obtained by subtracting the value of the difference time calculated at step S120 from the current date/time acquired at step S41 (step S121). Then, the measurement date/time set in this manner is recorded in the measurement result recording unit 260 (step S122). In the example illustrated in FIG. 29A, the measurement dates/times of the measurement results of the sequence numbers 30 to 28 are specified as 10:00:00, 9:36:00 and 9:13:00 on July 5 as a result of subtraction of the difference times 0, 1440 and 2820 from 10:00:00 on July 5, which is the current date/time.

A case where it is determined at step S119 that Ch=Chp does not hold is a case where the battery replacement is performed at least once after the measurement of the measurement result acquired at step S118. In this case, the control unit 240 searches the measurement results recorded in the measurement result recording unit 260 for a measurement result having the same battery replacement count information as the measurement result acquired at step S118 (step S123), and determines whether there is a measurement result with the same battery replacement count information and a record of a valid measurement date/time (step S124). In the example illustrated in FIG. 29B, the battery replacement count of the measurement results of the sequence numbers 1 to 27 is not 2, and therefore the measurement result recorded in the measurement result recording unit 260 is retrieved. For the measurement result of the battery replacement count of 0, it is determined that there is the measurement result of the sequence number 1 as a measurement result for which a valid measurement date/time is specified, and for the measurement result of the battery replacement count of 1, it is determined that there is the measurement result of the sequence number 25 as a measurement result for which a valid measurement date/time is specified.

When it is determined to be No at step S124, the measurement date/time cannot be specified for the measurement result acquired at step S118, and therefore “invalid” is set as the measurement date/time (step S125), and, the process proceeds to step S122.

When it is determined to be Yes at step S124, the control unit 240 acquires, as reference data, a measurement result with the same battery replacement count information as that of the measurement result acquired at step S118 and a record of a valid measurement date/time from among the measurement results recorded in the measurement result recording unit 260 (step S126). When there is a plurality of measurement results that can be reference data, for example, the measurement result with the smallest sequence number is selected, but the selection condition is not limited to this because it suffices to select any one of them for the process described later. In the example illustrated in FIG. 29B, the measurement result of the sequence number 1 is acquired as reference data from the measurement results of the battery replacement count of 0, and the measurement result of the sequence number 25 is acquired as reference data from the measurement results of the battery replacement count of 1.

Next, the control unit 240 determines whether the count value of the RTC at the time of measurement of the reference data is not greater than the count value of the RTC at the time of measurement of the measurement result acquired at step S118 (step S127).

When it is determined to be Yes at step S127, as the difference time, the control unit 240 sets a value obtained by subtracting the count value of the RTC at the time of measurement of the reference data from the count value of the RTC at the time of measurement of the measurement result acquired at step S118 (step S128). Then, the control unit 240 acquires the measurement date/time of the reference data (step S129), and sets a value obtained by adding the difference time set at step S128 to the measurement date/time acquired at step S129 as the measurement date/time of the measurement result acquired at step S118 (step S130). Then, the process proceeds to step S122. In the example illustrated in FIG. 29B, in each of the measurement results of the sequence numbers 20 to 23 and the sequence numbers 26 and 27, the count value of the RTC of the reference data is not greater than the count value of the RTC at the time of measurement. Accordingly, by subtracting the count value 4722237 of the reference data from the count values of the RTC at the time of measurement of the measurement results of the sequence numbers 20 to 23, 41760, 43200, 44640 and 46080 are obtained as difference times. Then, by adding these difference times to 20:10:00 on June 17 as the measurement dates/times of the reference data, the measurement dates/times of the measurement results of the sequence numbers 20 to 23 are specified as 7:46:00, 8:10:00, 8:34:00 and 8:58:00 on June 18. In addition, by subtracting the count value 1440 of the reference data from the count values of the RTC at the time of measurement of the measurement results of the sequence numbers 26 and 27, 1440 and 2880 are obtained as difference times. By adding these difference times to 10:48:00 on July 3 as the measurement date/time of the reference data, the measurement dates/times of the measurement results of the sequence numbers 26 and 27 are specified as 11:12:00 and 11:36:00 on July 3.

When it is determined to be No at step S127, the control unit 240 sets, as the difference time, a value obtained by subtracting the count value of the RTC at the time of measurement of the measurement result acquired at step S118 from the count value of the RTC at the time of measurement of the reference data (step S131). Then, the control unit 240 acquires the measurement date/time of the reference data (step S132), and sets a value obtained by subtracting the difference time set at step S131 from the measurement date/time acquired at step S132 as the measurement date/time of the measurement result acquired at step S118 (step S133). Then, the process proceeds to step S122. In the example illustrated in FIG. 29B, the count value 1440 of the RTC of the reference data is greater than the count value 60 of the RTC at the time of measurement of the measurement result of the sequence number 24. Accordingly, by subtracting the count value 60 of the RTC at the time of measurement of the measurement result of the sequence number 24 from the count value 1440 of the RTC at the time of measurement of the reference data, 1380 is obtained as a difference time. Then, by subtracting that difference time from 10:48:00 on July 3 as the measurement date/time of the reference data, the measurement date/time of the measurement result of the sequence number 24 is specified as 10:25:00 on July 3.

It is to be noted that as the processes after the acquisition of the reference data at step S126, when the count value of the RTC at the time of measurement of the reference data is greater than the count value of the RTC at the time of measurement of the measurement result acquired at step S118, a value (i.e., negative value) obtained by subtracting the count value of the RTC at the time of measurement of the reference data from the count value of the RTC at the time of measurement of the measurement result acquired at step S118 may be set as the difference time, and a value obtained by adding the difference time to the measurement date/time of the reference data may be set (i.e., a date/time prior to the measurement date/time of the reference data may be set) as the measurement date/time of the measurement result acquired at step S118. In this way, the processes of step S131 to step S133 are equivalent to step S128 to step S130, and therefore the determination of step S127 can be omitted.

In this manner, according to the present embodiment, regardless of the number of the measurement results that have not been transmitted from the blood pressure monitor 10 to the smart phone 20, or have not been received at the smart phone 20 after a transmission from the blood pressure monitor 10 to the smart phone 20, the measurement date/time can be specified when even one measurement result with the same battery replacement count information and a specified valid measurement date/time is recorded in the measurement result recording unit 260 of the smart phone 20. In addition, the processing load of the blood pressure monitor 10 is small because the differential processing of the count value of the RTC is not performed in the blood pressure monitor 10.

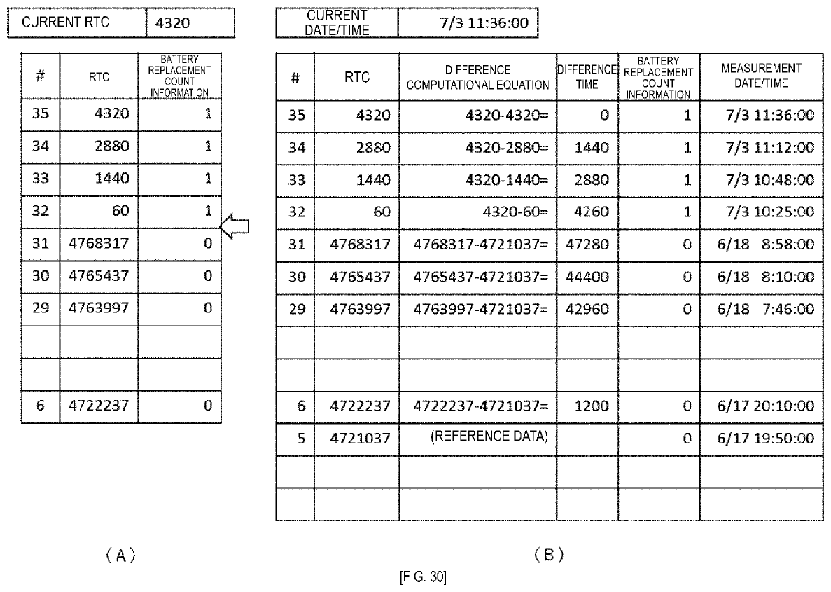

FIG. 30A is a diagram illustrating another example of a data configuration of the measurement result recording unit 180 in the blood pressure monitor 10 according to the present embodiment. FIG. 30B is a diagram illustrating a detailed example of a process of specifying a measurement date/time and another example of a part of a data configuration in the smart phone 20 according to the present embodiment.

In this example, since the measurement of blood pressure information has been performed at the blood pressure monitor 10 for 35 cases, greater than 30 cases that is the recordable cases of the measurement result recording unit 180, the data in the measurement result recording unit 180 is overwritten, and the measurement results of the sequence number 5 or smaller are not recorded. In addition, the measurement results of the sequence numbers 6 to 35 illustrated in FIG. 30A are cases where the measurement results have not been transmitted to the smart phone 20, or not been received at the smart phone 20 after a transmission from the blood pressure monitor 10. It should be noted that the smart phone 20 has received the measurement result of the sequence number 5, and in this measurement result, the battery replacement count information is 0 and a valid measurement date/time, 19:50:00 on June 17, is specified.

Regarding the measurement result of the sequence number 6 in the example illustrated in FIG. 30A, when the measurement result recording unit 260 is searched at step S123 for a measurement result with battery replacement count information of 0, there is the measurement result of the sequence number 5, and it is therefore determined to be Yes at step S124. Then, with the measurement result of the sequence number 5 as a reference data and its count value 4721037 of the RTC at the time of measurement, the measurement date/time of the measurement result of the sequence number 6 is specified as 20:10:00 on June 17 in accordance with the processes of steps S128 to 130. Likewise, also for the measurement results of the sequence numbers 29 to 31, the measurement dates/times are specified (the same applies to the measurement results of the sequence numbers 7 to 28 omitted in the drawing). For the measurement results of the sequence numbers 32 to 35, the measurement dates/times are specified in accordance with the processes of step S119 to step S121. The measurement dates/times of the measurement results of the sequence numbers 32 to 35 are specified in accordance with steps S120 to 122, but the description thereof is omitted.

In this manner, even in the case where the measurement result is not transmitted at the blood pressure monitor 10 and the measurement result recorded in the measurement result recording unit 180 is overwritten, the measurement date/time can be specified when there is a measurement result with the same battery replacement count information and a specified valid measurement date/time in the smart phone 20.

In the present embodiment, the difference between the count value of the RTC at the time of measurement of the blood pressure information and the count value of the RTC at the time of transmission is not calculated at the blood pressure monitor 10. However, for measurement results whose battery replacement count information at the time of measurement of the blood pressure information matches the latest battery replacement count information at the time of transmission, the blood pressure monitor 10 may calculate the difference of the count value of the RTC and transmit the calculated difference to the smart phone 20 together with the blood pressure information.

In the present embodiment, when a measurement result with the same battery replacement count information as the battery replacement count information included in the measurement result transmitted from the blood pressure monitor 10 is not accumulated in the measurement result recording unit 260, the measurement date/time cannot be specified, and the measurement date/time is set as “invalid”. For measurement results whose measurement date/time is set as “invalid” in the above-mentioned manner, the measurement result whose measurement date/time is set as “invalid” may be utilized in the aspect illustrated in FIG. 14 as described in the exceptional case 2 of the first embodiment. The case where the battery replacement count information is used is not limited to the case where the measurement date/time is set as “invalid” for all measurement results of one battery replacement count, and may be a case where the measurement date/time is set as “invalid” for all measurement results of a plurality of battery replacement counts. The following will describe a case where the measurement date/time is set as “invalid” for all measurement results of a battery replacement count of 2 and all measurement results of a battery replacement count of 3. The same applies to a case where there is three or more battery replacement counts whose measurement date/time is set as “invalid” for all measurement results.

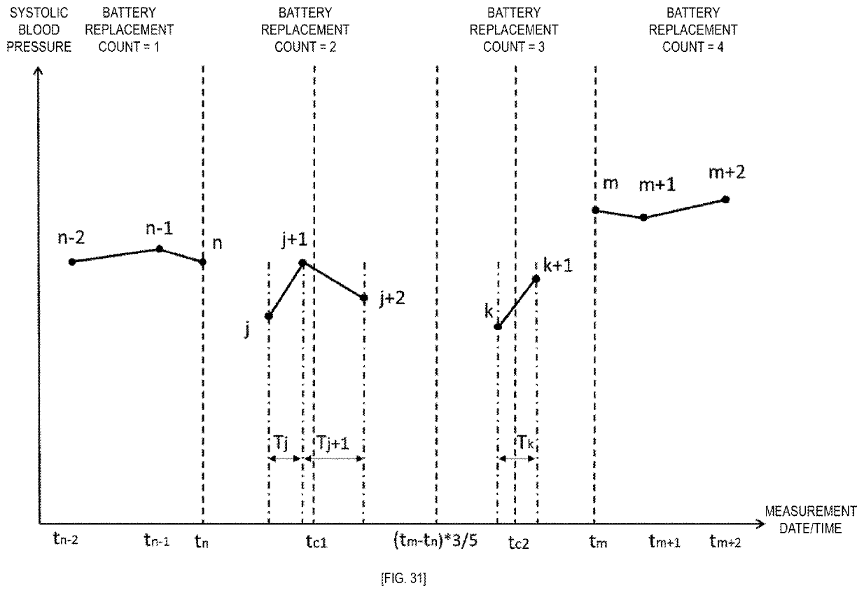

FIG. 31 illustrates an example of a display of a change with time of a systolic blood pressure as blood pressure information.

Assume here that for measurement results of the sequence number n or smaller whose battery replacement count is 1 and the sequence number m or greater whose battery replacement count is 4, measurement dates/times of tₙ, tₘ and the like are specified. On the other hand, for measurement results of the sequence numbers j to j+2 whose battery replacement count is 2, the intervals Tⱼ and Tⱼ₊₁ of the measurement dates/times of the sequence numbers j to j+1 and the sequence numbers j+1 to j+2, respectively, are specified by the count value of the RTC. Likewise, also for measurement results of the sequence numbers k to k+1 whose battery replacement count is 3, the interval Tₖ of the measurement date/time is specified by the count value of the RTC. Therefore, the intervals in the time direction of the data corresponding to these sequence numbers are set to correspond to the intervals of the respective count values of the RTC. Then, in the case where a temporary graph of measurement results whose measurement date/time is set as “invalid” is disposed in a graph representing a time-series change of measurement results, the interval between the valid measurement dates/times before and after the measurement results whose measurement date/time is set as “invalid” is divided by a predetermined ratio with respect to the battery replacement count of the measurement result whose measurement date/time is “invalid”. Thus, the temporary graph of the measurement results whose measurement date/time is set as “invalid” can be disposed such that the centers t_{c1} and t_{c2} in the time direction of the measurement results whose measurement date/time is set as “invalid” match respective center values of the divided plurality of time intervals. In FIG. 31, the interval between the valid measurement dates/times tₘ and tₙ before and after the measurement results whose measurement date/time is set as “invalid” is divided by a ratio corresponding to the number of points of the measurement results of respective battery replacement counts. Specifically, there are three measurement results whose battery replacement count is 2 and two measurement results whose battery replacement count is 3, and accordingly the region where the temporary graph of the measurement results of each battery replacement count is disposed is divided by the time points (tₘ−tₙ)*3/5 obtained by dividing tₘ−tₙ by 3:2. Then, the center t_{c1} of the temporary graph of the measurement results of the sequence numbers j to j+2 is disposed at t_{c1}={(tₘ−tₙ)*3/5−tₙ}/2, and the center t_{c2} of the temporary graph of the measurement results of the sequence numbers k to k+1 is disposed at t_{c2}={tₘ−(tₘ−tₙ)*3/5}/2.

In this manner, even for a measurement result whose exact measurement date/time cannot be specified, it is possible to know a relative time-series change with other measurement results whose measurement date/time can be specified, thus providing useful information for health management.

The method of disposing the temporary graph of the measurement result of the measurement date/time is set as “invalid” in a graph representing a time-series change of measurement results is not limited to the above-described method. For example, while the interval between the tₘ and tₙ, which are valid measurement dates/times before and after the measurement results that are invalid, is divided by a ratio corresponding to the number of points of the measurement results of each battery replacement count, tₘ−tₙ may be divided in accordance with the width in the time direction of the count values of the RTC of the measurement results of each battery replacement count. In the example illustrated in FIG. 31, the width in the time direction of the count values of the RTC of the measurement results whose battery replacement counts are 2 is Tⱼ+Tⱼ₊₁, and the width in the time direction of the count values of the RTC of the measurement results whose battery replacement counts are 3 is Tₖ. Accordingly, it is divided by time points obtained by dividing tₘ−tₙ by a ratio of (Tⱼ+Tⱼ₊₁):Tₖ into a plurality of time intervals for disposing the temporary graph of the measurement results of each battery replacement count. Thus, the temporary graph of the measurement result whose measurement date/time is set as “invalid” can be disposed such that the centers t_{c1} and t_{c2} in the time direction of the measurement results whose measurement date/time is “invalid” match the respective center values of the plurality of time intervals divided in the above-described manner.

Other Points

The description of each example described above is merely illustrative of the present invention, and the present invention is not limited to the specific embodiments described above. Within the scope of the technical idea of the present invention, various modifications and combinations may be made.

For example, the measurement unit may be a body information measurement apparatus other than a blood pressure monitor, such as a weight scale, a body composition meter, a pulse meter, or a thermometer. Further, the measurement unit may be an activity meter that measures the exercise amount in a pedometer, a tread mill, an Aerobike (trade name), or the like. In this case, the measured quantity to be displayed on the display unit may be the number of steps, a running (walking) distance, and the like, a value such as the estimated heat consumption, or both of them. In addition, the measurement unit may be an environment sensor device that measures environmental information such as room temperature, humidity, noise, and illuminance.

In addition, the device including the measurement date/time calculation unit is not limited to a smart phone, and may be another mobile information terminal such as a tablet terminal, or may be a stationary terminal.

### REFERENCE NUMERALS LIST

1 Health information management system
10 Blood pressure monitor
130, 210 Communication unit
150, 240 Control unit
170, 230 Storage unit
180, 260 Measurement result recording unit
20 Smart phone

1. A measurement apparatus comprising:
a monitor including a clock configured to measure an elapsed time from predetermined timing as a count value, a first memory configured to plurally accumulate a first measurement result including measurement information acquired through measurement, identification information uniquely assigned to the measurement information in order of acquisition of the measurement information and the count value at a time of measurement, and a transmitter configured to transmit the first measurement result accumulated in the first memory;
a receiver configured to receive the first measurement result from the monitor;
a second memory configured to accumulate a second measurement result including at least date/time information at the time of the measurement and the measurement information included in the first measurement result received from the monitor; and
a processor configured to calculate a measurement date/time at which the measurement information is acquired by subtracting a difference between the count value at a reference date/time and the count value at the time of the measurement from the reference date/time for the first measurement result, the reference date/time being a time point when communication with the monitor is established, wherein
the monitor stores power recovery information related to a loss and a recovery of power at the monitor; and
the processor determines whether the measurement information included in the first measurement result is acquired before the loss and the recovery of the power on a basis of the power recovery information received from the monitor, and, when the measurement information is acquired before the loss and the recovery of the power, the processor calculates the measurement date/time of the measurement information corresponding to the first measurement result by adding, to the measurement date/time included in the second measurement result that is finally accumulated in the second measurement result accumulated in the second memory and for which the measurement date/time has been calculated, a difference between the count value at the time of the measurement corresponding to the second measurement result that is finally accumulated and the count value at the time of the measurement corresponding to the first measurement result.

2. The measurement apparatus according to claim 1, wherein the monitor calculates the difference, and transmits the difference to the processor.

3. The measurement apparatus according to claim 1, wherein the difference is calculated at the processor on a basis of the count value at the reference date/time and the count value at the time of the measurement transmitted from the monitor.

4. The measurement apparatus according to claim 2, wherein
at a time of a first power recovery after the power loss, the monitor assigns a power recovery flag as power recovery information to all the first measurement results accumulated in the first memory; and
the processor determines that the measurement information included in the first measurement result assigned with the power recovery flag has been acquired before the power loss.

5. The measurement apparatus according to claim 2, wherein
at a first power recovery after the power loss, the monitor assigns a power recovery flag that is the power recovery information to a latest first measurement result in the first measurement results accumulated in the first memory;
the monitor transmits the first measurement result accumulated in the first memory in order from a latest one; and
the processor determines that the measurement information included in the first measurement result assigned with the power recovery flag and the first measurement result received after the first measurement result assigned with the power recovery flag has been acquired before the power loss.

6. The measurement apparatus according to claim 2, wherein
at a first power recovery after the power loss, the monitor stores, as reference identification information, the identification information included in the measurement information included in a latest first measurement result in the first measurement results accumulated in the first memory, and when it is indicated that the identification information assigned to the measurement result is acquired at a time point before the reference identification information as a result of comparison between the identification information assigned to each measurement information and the reference identification information at a time of transmission of the first measurement result, the monitor assigns a power recovery flag as the power recovery information to the measurement information and performs the transmission; and
for a measurement result assigned with the power recovery flag, the processor determines that the measurement information that is received has been acquired before the power loss.

7. The measurement apparatus according to claim 2, wherein
at a first power recovery after the power loss, the monitor stores, as reference identification information, the identification information included in the measurement information included in a latest first measurement result in the first measurement results accumulated in the first memory, and when, at transmission of the first measurement result in descending order of the identification information from the latest first measurement result in the first measurement results accumulated in the first memory, the identification information included in the first measurement result to be transmitted is identical to the reference identification information as a result of comparison between the identification information included in the first measurement result to be transmitted and the reference identification information, the monitor assigns a power recovery flag that is the power recovery information to the first measurement result to be transmitted and performs the transmission; and
the processor determines that the measurement information included in the first measurement result assigned with the power recovery flag and the first measurement result received after the first measurement result assigned with the power recovery flag has been acquired before the power loss.

8. The measurement apparatus according to claim 2, wherein
at a first power recovery after the power loss, the monitor stores, as reference identification information that is the power recovery information, the identification information of a latest first measurement result in the first measurement results accumulated in the first memory, and transmits the reference identification information at a time of transmission of the first measurement result; and
when it is indicated that the identification information of the first measurement result that is received is acquired at a time point before the reference identification information as a result of comparison between the identification information and the reference identification information, the processor determines that the measurement information included in the first measurement result has been acquired before the power loss.

9. The measurement apparatus according to claim 2, wherein
when the count value at the time of the measurement corresponding to the second measurement result finally accumulated in the second memory is not accumulated in the first memory, or when it is indicated that the count value at the time of the measurement corresponding to the first measurement result is before the count value at a time of one previous measurement of the first measurement result, the monitor or the processor sets the difference as invalid; and
for the first measurement result that is acquired before the power loss and is measured on and after the measurement of the first measurement result whose difference is invalid, the processor sets the measurement date/time as invalid and performs accumulation in the second .

10. The measurement apparatus according to claim 9, wherein the processor is further configured to display the measurement information in a time-series manner, wherein
for the second measurement result whose measurement date/time is invalid, the processor generates a temporary graph in which the measurement information of the second measurement result assigned with the identification information that is successive is disposed in a time-series manner in accordance with the difference of the count value at the time of the measurement between the measurement information assigned with the identification information that is successive, and disposes the temporary graph such that a center in a time direction of the temporary graph matches a center in the time direction between the measurement dates/times that are not invalid immediately before and after the second measurement result whose measurement date/time is invalid.

11. A measurement apparatus comprising:
a monitor including a clock configured to measure an elapsed time from predetermined timing as a count value, a first memory configured to plurally accumulate a first measurement result including measurement information acquired through measurement, identification information uniquely assigned to the measurement information in order of acquisition of the measurement information and the count value at a time of measurement, and a transmitter configured to transmit the first measurement result accumulated in the first memory;
a receiver configured to receive the first measurement result from the monitor;
a second memory configured to accumulate a second measurement result including at least date/time information at the time of the measurement and the measurement information included in the first measurement result received from the monitor; and
a processor configured to calculate a measurement date/time at which the measurement information is acquired by subtracting a difference between the count value at a reference date/time and the count value at the time of the measurement from the reference date/time for the first measurement result, the reference date/time being a time point when communication with the monitor is established, wherein
the monitor stores power recovery history information related to a history of a loss and a recovery of power at the monitor;
the first memory accumulates the first measurement result including the power recovery history information at the time of the measurement;
the second memory accumulates the first measurement result including the power recovery history information received from the monitor; and
when the power recovery history information at a time of acquisition of the measurement information included in the first measurement result received from the monitor is identical to the power recovery history information that is latest in the power recovery history information, the processor calculates the measurement date/time at which the measurement information is acquired by subtracting the difference between the count value at the reference date/time and the count value at the time of the measurement from the reference date/time for the first measurement result, the reference date/time being a time point when a communication with the monitor is established.

12. The measurement apparatus according to claim 11, wherein the monitor calculates the difference, and transmits the difference to the processor.

13. The measurement apparatus according to claim 11, wherein the difference is calculated at the processor on a basis of the count value at the reference date/time and the count value at the time of the measurement transmitted from the monitor.

14. The measurement apparatus according to claim 12, wherein the processor determines a power recovery history at the time of the acquisition of the measurement information included in the first measurement result on a basis of the power recovery history information received from the monitor, and the processor calculates the measurement date/time of the measurement information corresponding to the first measurement result through addition or subtraction of the difference between the count value at the time of the measurement corresponding to the second measurement result and the count value at the time of the measurement corresponding to the first measurement result with respect to the measurement date/time included in any of the second measurement results having a power recovery history identical to the power recovery history that is determined.

15. The measurement apparatus according to claim 14, wherein the processor determines the power recovery history at the time of the acquisition of the measurement information included in the first measurement result on the basis of the power recovery history information received from the monitor, and, when no valid measurement date/time has been calculated for any of the second measurement results having the power recovery history identical to the power recovery history that is determined, the processor sets the measurement date/time as invalid and performs accumulation in the second memory for the first measurement result.

16. The measurement apparatus according to claim 15, wherein the processor is further configured to display the measurement information in a time-series manner, wherein
for the second measurement result whose measurement date/time is invalid, the processor generates a temporary graph in which the measurement information of the second measurement result assigned with the identification information that is successive is disposed in a time-series manner in accordance with the difference of the count value at the time of the measurement between the measurement information assigned with the identification information that is successive, and disposes the temporary graph such that a center in a time direction of the temporary graph matches a center in the time direction between the measurement dates/times that are not invalid immediately before and after the second measurement result whose measurement date/time is invalid.

17. The measurement apparatus according to claim 16, wherein when there is a plurality of the power recovery histories in which the measurement date/time is invalid for all the second measurement results having the power recovery histories identical to each other, the processor generates a temporary graph in which the measurement information of the second measurement result assigned with the identification information that is successive is disposed in a time-series manner in accordance with the difference of the count value at the time of the measurement between the measurement information assigned with the identification information that is successive for the power recovery histories in which the measurement date/time is invalid for all the second measurement results, divides the time direction between the measurement dates/times that are not invalid immediately before and after the measurement result whose measurement date/time is invalid into a plurality of time intervals for each power recovery history, and disposes the temporary graph such that a center in a time direction of the temporary graph of the second measurement result whose measurement date/time is invalid of each power recovery history matches a center of the time interval corresponding to each power recovery history.
